(19) United States
(12) Patent Application Publication
(10) Pub. No.: US 2020/0276299 A1
(43) Pub. Date: Sep. 3, 2020

(54) LIPOSOMAL FORMULATIONS COMPRISING SAPONIN AND METHODS OF USE

(71) Applicant: Infectious Disease Research Institute, Seattle, WA (US)

(72) Inventors: Christopher B. FOX, Seattle, WA (US); Susan S. LIN, Seattle, WA (US)

(21) Appl. No.: 16/645,456

(22) PCT Filed: Sep. 7, 2018
(86) PCT No.: PCT/US2018/049832

§ 371 Date: Mar. 6, 2020

Related U.S. Application Data
(60) Provisional application No. 62/556,257, filed on Sep. 8, 2017.

(51) Int. Cl. A61K 39/21; A61K 47/24; A61K 39/04; A61K 39/015

(52) U.S. Cl. CPC A61K 39/21; A61K 47/24; A61K 2039/55572; A61K 39/015; A61K 39/04

(57) ABSTRACT

Provided herein are liposomal formulations containing saponin and optionally, a lipopolysaccharide. Also provided IC herein are pharmaceutical compositions and vaccine compositions comprising the liposomal formulations and an antigen. The pharmaceutical compositions and vaccine compositions are capable of eliciting or enhancing of an immune response, for example, for vaccine or therapeutic uses. Compositions and methods related to making the liposomal formulations and using the liposomal formulations for eliciting or enhancing an immune response are also provided.

### CROSS-REFERENCE TO RELATED APPLICATIONS

This application claims the benefit of priority of U.S. Provisional Application No. 62/556,257, filed Sep. 8, 2017, which is incorporated by reference here in in its entirety for any purpose.

### TECHNICAL FIELD

The present disclosure relates generally to pharmaceutical compositions and vaccine compositions. More specifically, embodiments described herein relate to liposomal formulations comprising a saponin and, optionally, a lipopolysaccharide (LPS) and methods of their manufacture.

### BACKGROUND

The immune system of higher organisms has been characterized as distinguishing foreign agents (or “non-self”) agents from familiar or “self” components, such that foreign agents elicit immune responses while “self” components are ignored or tolerated. Immune responses have traditionally been characterized as either humoral responses, in which antibodies specific for antigens are produced by differentiated B lymphocytes known as plasma cells, or cell mediated responses, in which various types of T lymphocytes act to eliminate antigens by a number of mechanisms. For example, CD4+ helper T cells that are capable of recognizing specific antigens may respond by releasing soluble mediators such as cytokines to recruit additional cells of the immune system to participate in an immune response. Also, CD8+ cytotoxic T cells that are also capable of specific antigen recognition may respond by binding to and destroying or damaging an antigen-bearing cell or particle. It is known in the immunological arts to provide certain vaccine compositions according to a variety of formulations, usually for the purpose of inducing a desired immune response in a host.

Several strategies for eliciting specific immune responses through the administration of a vaccine to a host include immunization with heat-killed or with live, attenuated infectious pathogens such as viruses, bacteria or certain eukaryotic pathogens; immunization with a non-virulent infective agent capable of directing the expression of genetic material encoding the antigen(s) to which an immune response is desired; and immunization with subunit vaccine compositions that contain isolated immunogens (such as proteins) from a particular pathogen in order to induce immunity against the pathogen. (See, e.g., Liu, 1998 Nature Medicine 4(5 suppl.):515.) For certain antigens there may be one or more types of desirable immunity for which none of these approaches has been particularly effective, including the development of vaccine compositions that are effective in protecting a host immunologically against human immunodeficiency viruses or other infectious pathogens, cancer, autoimmune disease, or other clinical conditions.

Various adjuvants have been employed in vaccine compositions in order to improve the immunogenicity associated with any given antigen while minimizing the potential for toxicity. For example, Quillaja saponins are a mixture of triterpene glycosides extracted from the bark of the tree Quillaja saponaria. Crude saponins have been employed as adjuvants in vaccine compositions against foot and mouth disease, and in amplifying the protective immunity conferred by experimental vaccine compositions against protozoal parasites such as Trypanosoma cruzi plasmodium and also the humoral response to sheep red blood cells (Bomford, Int. Arch. Allerg. appl. Immun., 67:127 (1982)). However, due to the heterogeneity and impurities present in crude mixtures, which affect adjuvant activity and toxicity, crude saponins are not desirable for use in veterinary practice or in pharmaceutical compositions for humans. Quil-A is a partially purified aqueous extract of the Quillaja saponin material, and is characterized chemically as a carbohydrate moiety in glycosidic linkage to the triterpenoid quillaic acid. While Quil-A presents an improvement over the crude saponins, it has also been shown to demonstrate considerable heterogeneity. QS21 is a HPLC-purified nontoxic fraction of Quil-A with adjuvant activity and its method of its production is disclosed (as QA21) in U.S. Pat. No. 5,057,540.

It has long been known that enterobacterial lipopolysaccharide (LPS) is a potent stimulator of the immune system, although its use in adjuvants has been curtailed by its toxic effects. A synthetic non-toxic derivative of the lipid A tail of LPS, Glucopyranosyl lipid A (GLA), however, is shown to have strong potential to induce immune responses as disclosed in U.S. Pat. No. 8,273,361. A naturally occurring non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of Bacterial Endotoxins, Plenum Publ. Corp., NY, p 407-419).

A further detoxified version of MPL results from the removal of the acyl chain from the 3-position of the disaccharide backbone, and is called 3-O-deacylated monophosphoryl lipid A (3D-MPL). It can be purified and prepared by the methods taught in GB 2122204B, which reference also discloses the preparation of diphosphoryl lipid A, and 3-O-deacylated variants thereof. For example, 3D-MPL has been prepared in the form of an emulsion having a small particle size less than 0.2 m in diameter, and its method of manufacture is disclosed in WO 94/21292. Aqueous formulations comprising monophosphoryl lipid A and a surfactant have been described in WO9843670A2.

Bacterial LPS-derived adjuvants to be formulated in adjuvant combinations may be purified and processed from bacterial sources, or alternatively they may be synthetic. For example, synthetic saponins and in particular, synthetic QS21 (SQS 21) have been disclosed (Ragupathi et al. Expert Rev Vaccines. 2011 April; 10(4): 463-470). Purified monophosphoryl lipid A is described in Ribi et at 1986 (supra), and 3-O-deacylated monophosphoryl or diphosphoryl lipid A derived from Salmonella sp. is described in GB 2220211 and U.S. Pat. No. 4,912,094. 3D-MPL and the 13(1-6) glucosamine disaccharides as well as other purified and synthetic lipopolysaccharides have been described (WO 98/01139; U.S. Pat. No. 6,005,099 and EP 0 729 473 B1, Hilgers et al., 1986 Int. Arch. Allergy Immunol., 79(4):392-6; Hilgers et at., 1987, Immunology, 60(1); 141-6; and EP 0 549 074 B1). In addition, a synthetic second-generation lipid adjuvant (SLA) designed by modification of GLA has been described (Paes et al. 2016, Vaccine, 34(35): 4123-4131).

Combinations of 3D-MPL and saponin derived from the bark of Quillaja Saponaria molina have been described in EP0761231B and US20080279926. WO 95/17210 discloses an adjuvant emulsion system based on squalene, α-tocopherol, and polyoxyethylene sorbitan monooleate (TWEEN™-80), formulated with QS21, and optionally including 3D-MPL. Despite the accessibility of such combinations, the use of adjuvants derived from natural products is accompanied by high production costs, inconsistency from lot to lot, difficulties associated with large-scale production, and uncertainty with respect to the presence of impurities in the compositional make-up of any given preparation.

Accordingly, there is a need for improved vaccine compositions, and in particular for vaccine compositions that beneficially contain high-purity, chemically defined adjuvant components that exhibit lot-to-lot consistency and that can be manufactured efficiently on an industrial scale without introducing unwanted or structurally undefined contaminants. The present disclosure fulfills these needs and offers other related advantages

### BRIEF SUMMARY OF THE INVENTION

The present disclosure in its several aspects is directed to compositions and methods that advantageously employ a saponin and optionally, a lipopolysaccharide as a component(s) in a liposomal formulation. In one aspect, the liposomal formulation includes a saponin and a lipopolysaccharide (LPS). In another aspect, the liposome formulation includes a saponin and does not contain a LPS. In another aspect, the liposomal formulation includes a saponin complexed to a sterol and, optionally, a LPS.

In certain embodiments there is provided a saponin comprising naturally derived and purified QS21 or synthetic QS21 (see, e.g., U.S. Pat. No. 5,057,540; EP 0 362 279 B1; WO 95/17210).

According to one embodiment of the disclosure described herein, the saponin is complexed to a sterol where the sterol is cholesterol.

The optional LPS used in the liposomal formulation of the disclosure can be selected from TLR4 agonists known and available in the art. In certain specific embodiments, the TLR4 agonist is selected from GLA, MPL, or 3D-MPL.

According to one aspect of the disclosure described herein, there are provided GLA compounds having the following structure:

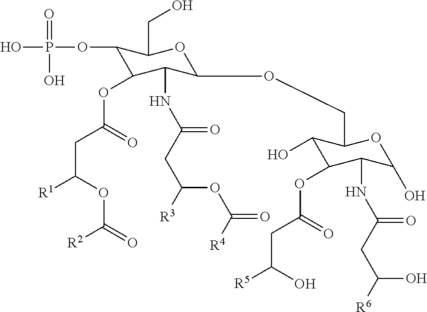

where R¹, R³, R⁵ and R⁶ are C₁₁-C₂₀ alkyl; and R² and R⁴ are C₉-C₂₀ alkyl. In some embodiments, R¹, R³, R⁵ and R⁶ are C₁₁₋₁₄ alkyl; and R² and R⁴ are C₁₂₋₁₅ alkyl. In some embodiments, R¹, R³, R⁵ and R⁶ are C₁₁ alkyl; and R² and R⁴ are C₁₃ alkyl. In some embodiments, R¹, R³, R⁵ and R⁶ are C₁₁ alkyl; and R² and R⁴ are C₉ alkyl. In some embodiments, R¹, R³, R⁵ and R⁶ are C₁₀ alkyl; and R² and R⁴ are C⁸ alkyl.

In some embodiments, GLA has the following structure and is referred to herein as SLA:

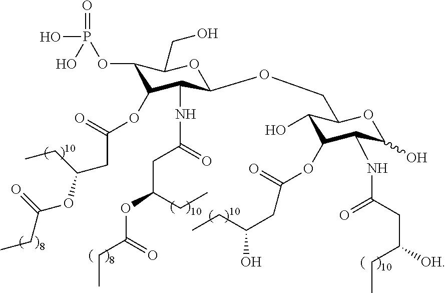

In some embodiments, GLA has the following structure (referred to in the examples as GLA*):

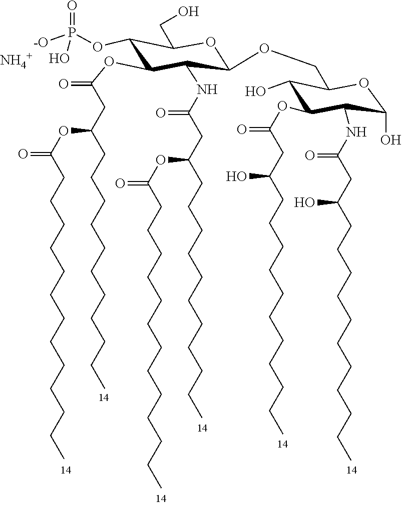

In certain embodiments of the disclosure described herein, there are provided an antigen that is associated with an infectious disease, cancer, or an autoimmune disease.

In another aspect, the disclosure provides methods for stimulating and enhancing an immune response against an antigen derived from or immunologically cross-reactive with at least one infectious pathogen that is associated with an infectious disease comprising administering to a mammal in need thereof a composition of the disclosure. In certain embodiments, the disclosure provides methods for eliciting and enhancing an immune response against at least one epitope, biomolecule, cell, or tissue that is associated with cancer. In certain embodiments, the disclosure provides methods for stimulating and enhancing an immune response against at least one epitope, biomolecule, cell, or tissue that is associated with an autoimmune disease. In certain embodiments, the disclosure provides methods for stimulating and enhancing an immune response against at least one epitope, biomolecule, cell, or tissue that is associated with an infectious disease

Also provided are methods of manufacturing the saponin containing liposomes of the present invention.

It is to be understood that one, some, or all of the properties of the various embodiments described herein may be combined to form other embodiments of the present disclosure. These and other aspects of the present disclosure will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1: This figure shows an exemplary manufacturing and scale-up process flow for exemplary formulations of the present invention

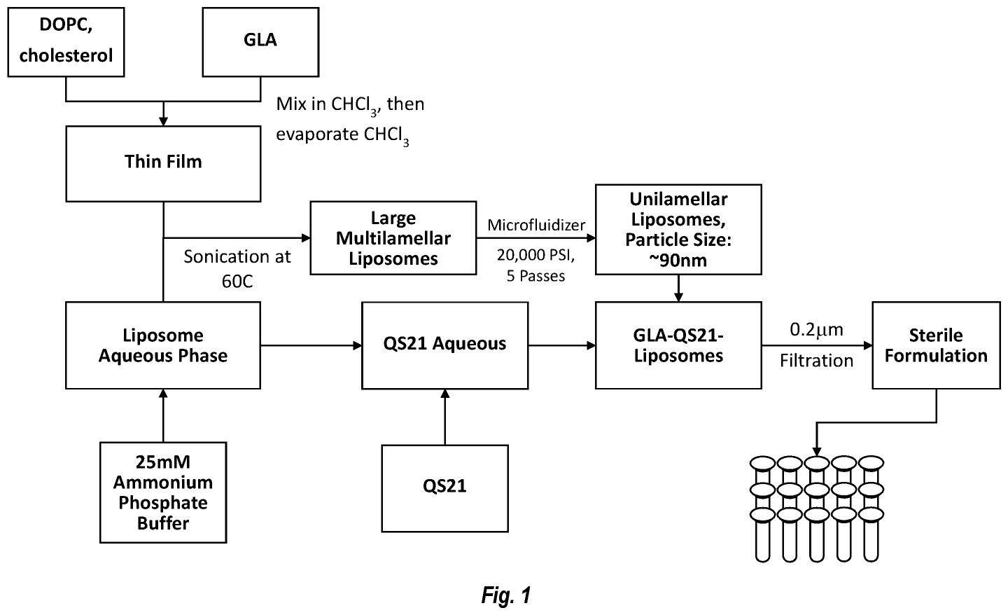

FIG. 2: This figure depicts the survival of the guinea pigs over time. A Mantel-Cox test was performed to determine significance with p, 0.05 indicating significance. The symbols along the lines are used solely to distinguish the lines and are not indicative of individual animals.

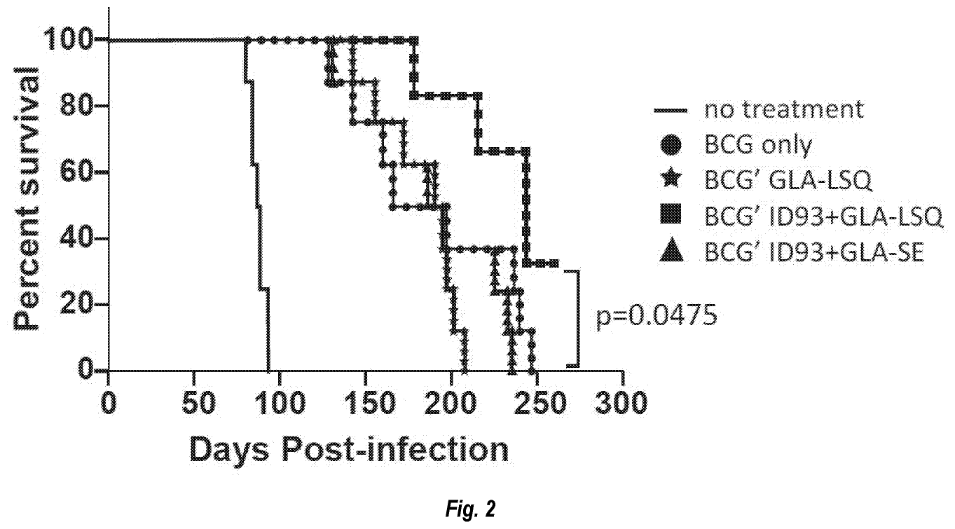

### DESCRIPTION OF THE INVENTION

The present disclosure is generally directed to liposomal formulations including a saponin and, optionally, a lipopolysaccharide (LPS), and related methods for using the same in pharmaceutical compositions and vaccine compositions. In certain aspects, the liposomal formulation may include a saponin complexed to a sterol and, optionally, a LPS. The pharmaceutical compositions and vaccine compositions of the disclosure include, for example,—liposomal formulations containing QS21 and, optionally, a GLA. In another example, the pharmaceutical compositions and vaccine compositions of the disclosure may include liposomal formulations containing QS21 complexed to a sterol and, optionally, a GLA. In specific preferred embodiments, the saponin is complexed to cholesterol.

The pharmaceutical compositions and vaccine compositions containing the liposomal formulation optionally further comprise an antigen where the antigen is associated with an infectious disease, cancer, or an autoimmune disease. The present disclosure also contemplates using the liposomal formulations as a pharmaceutical composition or vaccine composition to elicit or enhance an immune response in a subject having an infectious disease, cancer, or an autoimmune disease.

There is an increasingly limited global availability of Quillaja saponaria Molina bark, suggesting that this natural resource may not be sufficient for large scale production of vaccine compositions that employ a high concentration of saponin in each dose (Ragupathi et al., Expert Rev. Vaccines 2011; 10(4):463-470. Furthermore, the expensive cost associated with the procurement of natural saponin is a limiting factor in its widespread use despite its potent adjuvant activity. In contrast, the liposomal formulations, pharmaceutical compositions and vaccine compositions provided herein advantageously use saponin in a low concentration range per dose compared to previous saponin-containing formulations known in the art. The compositions of the present disclosure thus beneficially contain high-purity, chemically defined components that exhibit lot-to-lot consistency and can be manufactured efficiently on an industrial scale.

### I. Definitions

The following terms have the following meanings unless otherwise indicated. Any undefined terms have their art recognized meanings.

As used herein and in the appended claims, the singular forms “a”, “an” and “the” include plural reference unless the content clearly dictates otherwise.

It is understood that aspect and embodiments of the disclosure described herein include “comprising,” “consisting,” and “consisting essentially of” aspects and embodiments.

In the present description, the terms “about” and “consisting essentially of” mean ±20% of the indicated range, value, or structure, unless otherwise indicated.

The use of the alternative (e.g., “or”) should be understood to mean either one, both, or any combination thereof of the alternatives.

As used herein, the terms “include,” “have” and “comprise” are used synonymously, which terms and variants thereof are intended to be construed as non-limiting.

The term “macromolecule” as used herein refers to large molecules exemplified by, but not limited to, peptides, proteins, oligonucleotides and polynucleotides of biological or synthetic origin.

The term “alkyl” means a straight chain or branched, noncyclic or cyclic, unsaturated or saturated aliphatic hydrocarbon containing the indicated number of carbon atoms. Unsaturated alkyls contain at least one double or triple bond between adjacent carbon atoms.

The terms “polypeptide”, “peptide”, and “protein” are used interchangeably herein to refer to polymers of amino acids of any length. The polymer may be linear or branched, it may comprise modified nucleotides or amino acids, and it may be interrupted by non-nucleotides or non-amino acids. The terms also encompass a nucleotide or amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polynucleotides or polypeptides containing one or more analogs of a nucleotide or an amino acid (including, for example, unnatural amino acids, etc.), as well as other modifications known in the art.

The term “isolated” means the molecule has been removed from its natural environment.

“Purified” means that the molecule has been increased in purity, such that it exists in a form that is more pure than it exists in its natural environment and/or when initially synthesized and/or amplified under laboratory conditions. Purity is a relative term and does not necessarily mean absolute purity.

A “polynucleotide” or “nucleic acid,” as used interchangeably herein, refer to polymers of nucleotides of any length, include DNA and RNA. The nucleotides can be, for example, deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

An “individual” or a “subject” is any mammal. Mammals include, but are not limited to humans, primates, farm animals, sport animals, pets (such as cats, dogs, horses), and rodents.

The practice of the present disclosure will employ, unless otherwise indicated, conventional techniques of molecular biology, recombinant DNA, biochemistry, and chemistry, which are within the skill of the art. Such techniques are explained fully in the literature. See, e.g., Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); B. Perbal, A Practical Guide To Molecular Cloning (1984); the treatise, Methods In Enzymology (Academic Press, Inc., N.Y.); and in Ausubel et al., Current Protocols in Molecular Biology, John Wiley and Sons, Baltimore, Md. (1989).

### II. Liposomal Formulations

The disclosure provides for liposomal formulations. The liposomal formulations comprise a saponin and, optionally, a LPS. Additionally, the liposomal formulations may optionally include at least one sterol and at least one phospholipid.

### A. Adjuvant

As discussed herein, the liposomal formulations of the disclosure comprise a saponin and, optionally, a LPS. Saponin and LPS are generally known to possess adjuvant activity.

Saponin

Saponins are taught in, e.g., U.S. Pat. No. 6,544,518; Lacaille-Dubois, M and Wagner H. (1996 Phytomedicine 2:363-386), U.S. Pat. No. 5,057,540, Kensil, Crit Rev Ther Drug Carrier Syst, 1996, 12 (1-2):1-55, and EP 0 362 279 B1. Particulate structures, termed Immune Stimulating Complexes (ISCOMS), comprising fractions of Quil A (saponin) are haemolytic and have been used in the manufacture of vaccine compositions (Morein, B., EP 0109942 B1). These structures have been reported to have adjuvant activity (EP 0 109 942 B1; WO 96/11711). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccine compositions. Use of QS21 is further described in Kensil et al. (1991. J. Immunology 146:431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711. Other saponins which have been used in systemic vaccination studies include those derived from other plant species such as Gypsophila and Saponaria (Bomford et al., Vaccine, 10(9):572-577, 1992).

In one embodiment of the liposomal formulation provided herein, the saponin is an immunologically active saponin fraction derived from the bark of Quillaja saponaria Molina. In one such embodiment, the saponin fraction is QS21.

Due to an increasingly limited global supply of Quillaja saponaria Molina bark and the challenges associated with achieving a highly purified immunologically active saponin fraction with batch-to-batch consistency, chemical production of synthetic saponins, such as synthetic QS21 (SQS21), QS21-Api, and QS21-Xyl, has been described (Ragupathi et al. Expert Rev Vaccines. 2011 April; 10(4): 463-470). Synthetic QS21 (SQS 21) and naturally derived QS21 have been shown to possess similar adjuvant activity.

In certain embodiments of the liposomal formulation provided herein, the saponin is synthetic. In one such embodiment, the synthetic saponin is synthetic QS21 (SQS21).

Escin is another compound related to the saponins that may be used in the embodiments of the liposomal formulations disclosed herein. Escin is described in the Merck index (12th Ed.: entry 3737) as a mixture of saponin occurring in the seed of the horse chestnut tree, Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin (also known as aescin) have been purified and shown to be biologically active (Yoshikawa M, et al. (Chem Pharm Bull (Tokyo) 1996 August; 44(8): 1454-1464)). Digitonin, which is also being described in the Merck index (12th Ed., entry 3204) as a saponin, is derived from the seeds of Digitalis purpurea and purified according to the procedure described by Gisvold et al., J. Am. Pharm. Assoc., 1934, 23, 664; and Rubenstroth-Bauer, Physiol. Chem., 1955, 301, 621

In certain illustrative embodiments, the saponin comprises Quil-A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the liposomal formulations of the present disclosure, for example combinations of at least two of the following group comprising QS21, QS7, Quil-A, escin, or digitonin.

It is contemplated herein that the liposomal formulation comprising a saponin and, optionally, a LPS is a composition for administration to a human subject. In certain embodiments, the concentration of saponin is from about 0.5 ug per dose to about 10 ug per dose or from about 1 μg per dose to about 10 μg per dose. In some preferred embodiments, the concentration of saponin is from about 0.5 μg per dose to about 8 μg per dose or from about 1 μg per dose to about 8 μg per dose. It will be understood by the skilled practitioner that if the concentration of a component is from about 0.5 ug per dose to about 10 ug per dose, the amount to be delivered to a subject will be from about 0.5 ug to about 10 ug per dose. The formulation itself may be diluted prior to delivery to the subject.

In certain illustrative embodiments of the composition provided herein, the concentration of saponin is about 1 μg per dose, about 2 μg per dose, about 3 μg per dose, about 4 μg per dose, about 5 μg per dose, about 6 μg per dose, about 7 μg per dose, about 8 μg per dose, about 9 μg per dose, or about 10 μg per dose. In some embodiments, the concentration of saponin is from about 1 μg per dose to about 2 μg per dose, about 2 μg per dose to about 3 μg per dose, about 3 μg per dose to about 4 μg per dose, about 4 μg per dose to about 5 μg per dose, about 5 μg per dose to about 6 μg per dose, about 6 μg per dose to about 7 μg per dose, about 7 μg per dose to about 8 μg per dose, about 8 μg per dose to about 9 μg per dose, or about 9 μg per dose to about 10 μg per dose. In some aspects, the saponin is at a concentration of less than about 1 μg per dose, e.g. from about 0.5 ug per dose to about 1 ug per dose.

### LPS

In exemplary embodiments of the present invention, the LPS is an immunostimulant. In other words, the LPS is capable of eliciting an immune response in a subject, either alone, or in combination with an antigen associated with a disease state. In certain illustrative embodiments, the LPS is a TLR4 agonist. As used herein, a “TLR4 agonist” refers to an agonist that affects its biological activities through its interaction with TLR4. In certain preferred embodiments, a TLR4 agonist used in the formulations of the disclosure is a glucopyranosyl lipid adjuvant (GLA), such as those described in U.S. Patent Publication Nos. US2007/021017, US2009/045033 and US2010/037466, the contents of which are incorporated herein by reference in their entireties.

As noted above, since GLA is chemically synthesized, it can be prepared in a substantially homogeneous form, which refers to a GLA preparation that is at least 80%, preferably at least 85%, more preferably at least 90%, more preferably at least 95% and still more preferably at least 96%, 97%, 98% or 99% pure with respect to the GLA molecule.

For example, in certain embodiments, the TLR4 agonist is a synthetic GLA having the following structure of Formula (I):

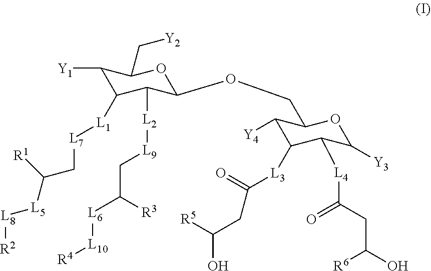

or a pharmaceutically acceptable salt thereof, where:
L₁, L₂, L₃, L₄, L₅ and L₆ are the same or different and independently —O—, —NH— or —(CH₂)—;
L₇, L₈, L₉, and L₁₀ are the same or different and independently absent or —C(═O)—;
Y₁ is an acid functional group;
Y₂ and Y₃ are the same or different and independently —OH, —SH, or an acid functional group;
Y₄ is —OH or —SH;
R¹, R³, R⁵ and R⁶ are the same or different and independently C₈₋₁₃ alkyl; and
R² and R⁴ are the same or different and independently C₆₋₁₁ alkyl.

In some embodiments of the synthetic GLA structure, R¹, R³, R⁵ and R⁶ are C₁₀ alkyl; and R² and R⁴ are C₈ alkyl. In certain embodiments, R¹, R³, R⁵ and R⁶ are C₁₁ alkyl; and R² and R⁴ are C₉ alkyl.

For example, in certain embodiments, the TLR4 agonist is a synthetic GLA having the following structure of Formula (II) or a pharmaceutically acceptable salt thereof:

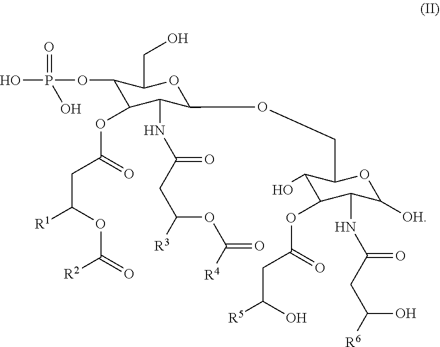

In certain embodiments of the above GLA structure, R¹, R³, R⁵ and R⁶ are C₁₁-C₂₀ alkyl; and R² and R⁴ are C₁₂-C₂₀ alkyl. In another specific embodiment, the GLA has the formula set forth above where R¹, R³, R⁵ and R⁶ are C₁₁ alkyl; and R² and R⁴ are C₁₃ alkyl. In another specific embodiment, the GLA has the formula set forth above where R¹, R³, R⁵ and R⁶ are C₁₀ alkyl; and R² and R⁴ are C₈ alkyl.

In another specific embodiment, the GLA has the formula set forth above where R¹, R³, R⁵ and R⁶ are C₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the TLR4 agonist is a synthetic GLA having the following structure of Formula (III) or a pharmaceutically acceptable salt thereof:

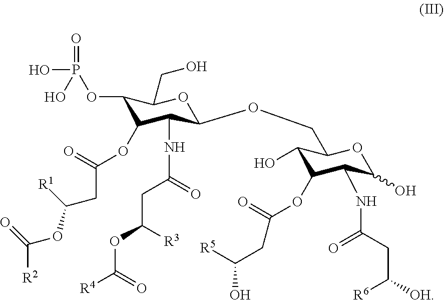

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the TLR4 agonist is a synthetic GLA having the following structure of Formula (IV):

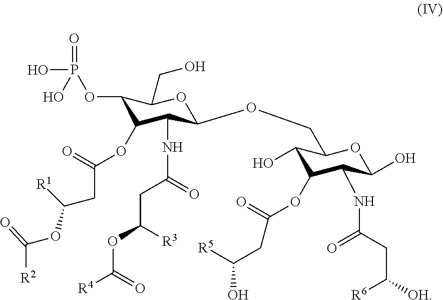

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the TLR4 agonist is a synthetic GLA having the following structure of Formula (V) or a pharmaceutically acceptable salt thereof:

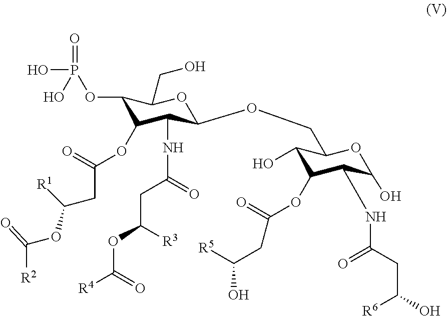

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the TLR4 agonist is a synthetic GLA having the following structure or a pharmaceutically acceptable salt thereof:

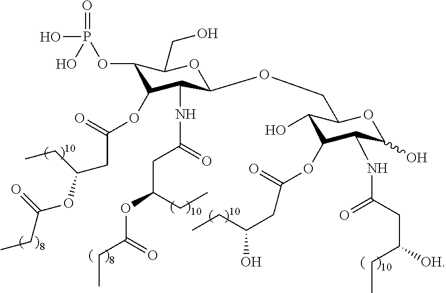

In certain embodiments, the TLR4 agonist is a synthetic GLA having the following structure (and referred to herein as SLA) or a pharmaceutically acceptable salt thereof:

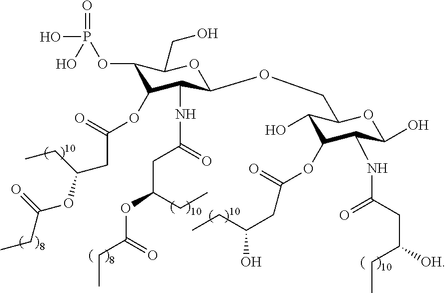

In certain embodiments, the TLR4 agonist is a synthetic GLA having the following structure or a pharmaceutically acceptable salt thereof:

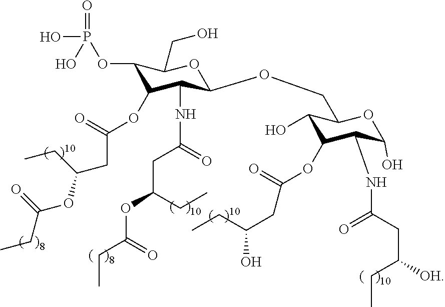

In an exemplary embodiment of the liposomal formulation provided herein, the LPS is GLA having the structure according to Formula (II), where R¹, R³, R⁵ and R⁶ are C11 alkyl; and R² and R⁴ are C13 alkyl, or a pharmaceutically acceptable salt thereof. In an exemplary embodiment of the liposomal formulation provided herein, the LPS is GLA having the structure according to Formula (II), where R¹, R³, R⁵ and R⁶ are C10 alkyl; and R² and R⁴ are C8 alkyl, or a pharmaceutically acceptable salt thereof. In still another exemplary embodiment of the liposomal formulation provided herein, the LPS is MPL.

In certain embodiments of the composition described herein, the LPS is a synthetic second-generation lipid adjuvant (SLA) designed by modification of GLA. In another embodiment, an attenuated lipid A derivative (ALD) is incorporated into the compositions described herein. ALDs are lipid A-like molecules that have been altered or constructed so that the molecule displays lesser or different of the adverse effects of lipid A. These adverse effects include pyrogenicity, local Shwarzman reactivity and toxicity as evaluated in the chick embryo 50% lethal dose assay (CELD₅₀). ALDs useful according to the present disclosure include monophosphoryl lipid A (MLA or MPL) and 3-deacylated monophosphoryl lipid A (3D-MLA or 3D-MPL). MLA (MPL) and 3D-MLA (3D-MPL) are known and need not be described in detail herein. See, for example, U.S. Pat. Nos. 4,436,727 and 4,912,094 incorporated herein by reference and for all purposes.

In the TLR4 agonist compounds above, the overall charge can be determined according to the functional groups in the molecule. For example, a phosphate group can be negatively charged or neutral, depending on the ionization state of the phosphate group.

### Synthesis of GLA Compounds

As mentioned above, the present disclosure provides GLA compounds. Representative GLA compounds of the present disclosure may be prepared by known organic synthesis techniques, see for example U.S. Pat. Nos. 8,722,064 and 8,273,361, incorporated herein by reference in their entirety and for all purposes.

The compounds of the present disclosure may generally be utilized as the free base or free acid. Alternatively, the compounds of this disclosure may be used in the form of acid or base addition salts. Acid addition salts of the free amino compounds of the present disclosure may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids.

Similarly, base addition salts of the acid compounds of the present disclosure may be prepared by methods well known in the art, and may be formed from organic and inorganic bases. Suitable organic bases include, but are not limited to, triethylamine and pyridine. Suitable inorganic bases include, but are not limited to, sodium hydroxide, potassium hydroxide, sodium carbonate, potassium carbonate, and ammonia. Thus, the term “pharmaceutically acceptable salt” of Formula (I) is intended to encompass any and all acceptable salt forms.

In addition, prodrugs are also included within the context of this disclosure. Prodrugs are any covalently bonded carriers that release a compound of Formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this disclosure where hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of Formula (I). Further, in the case of a carboxylic acid (COOH), esters may be employed, such as methyl esters, ethyl esters, and the like.

With regard to stereoisomers, the compounds of Formula (I) may have chiral centers and may occur as racemates, racemic mixtures and as individual enantiomers or diastereomers. All such isomeric forms are included within the present disclosure, including mixtures thereof. Furthermore, some of the crystalline forms of the compounds of Formula (I) may exist as polymorphs, which are included in the present disclosure. In addition, some of the compounds of Formula (I) may also form solvates with water or other organic solvents. Such solvates are similarly included within the scope of this disclosure.

It is contemplated herein that the liposomal formulation comprising a saponin and an LPS is a composition for administration to a human subject. In certain embodiments, the concentration of LPS is from about 1 μg per dose, about 2 μg per dose or about 2.5 μg per dose to about 25 μg per dose. In some preferred embodiments, the concentration of LPS is from about 3 μg per dose to about 20 μg per dose.

In certain illustrative embodiments of the composition provided herein, the concentration of LPS is about 2.5 μg per dose, about 3 μg per dose, about 3.5 μg per dose, about 4 μg per dose, about 4.5 μg per dose, about 5 μg per dose, about 5.5 μg per dose, about 6 μg per dose, about 6.5 μg per dose, about 7 μg per dose, about 7.5 μg per dose, about 8 μg per dose, about 8.5 μg per dose, about 9 μg per dose, about 9.5 μg per dose, about 10 μg per dose, about 10.5 μg per dose, about 11 μg per dose, about 11.5 μg per dose, about 12 μg per dose, about 12.5 μg per dose, about 13 μg per dose, about 13.5 μg per dose, about 14 μg per dose, about 14.5 μg per dose, about 15 μg per dose, about 15.5 μg per dose, about 16 μg per dose, about 16.5 μg per dose, about 17 μg per dose, about 17.5 μg per dose, about 18 μg per dose, about 18.5 μg per dose, about 19 μg per dose, about 19.5 μg per dose, about 20 μg per dose, about 20.5 μg per dose, about 21 μg per dose, about 21.5 μg per dose, about 22 μg per dose, about 22.5 μg per dose, about 23 μg per dose, about 23.5 μg per dose, about 24 μg per dose, about 24.5 μg per dose, or about 25 μg per dose.

In certain embodiments, the concentration of LPS is from about 2.5 μg per dose to about 5 μg per dose, about 5 μg per dose to about 7.5 μg per dose, about 7.5 μg per dose to about 10 μg per dose, about 10 μg per dose to about 12.5 μg per dose, about 12.5 μg per dose to about 15 μg per dose, about 15 μg per dose to about 17.7 μg per dose, about 17.5 μg per dose to about 20 μg per dose, about 20 μg per dose to about 22.5 μg per dose, or about 22.5 μg per dose to about 25 μg per dose.

### Ratio of Saponin to LPS

In an exemplary aspect of the liposomal formulation described herein, the ratio of saponin to LPS is about 1 to 2.5. In some embodiments having this ratio of saponin to LPS, the concentration of saponin is about 1 μg per dose and the concentration of LPS is about 2.5 μg per dose. In some embodiments having this ratio of saponin to LPS, the concentration of saponin is about 2 μg per dose and the concentration of LPS is about 5 μg per dose. In some embodiments having this ratio of saponin to LPS, the concentration of saponin is about 3 μg per dose and the concentration of LPS is about 7.5 μg per dose. In some embodiments having this ratio of saponin to LPS, the concentration of saponin is about 4 μg per dose and the concentration of LPS is about 10 μg per dose. In some embodiments having this ratio of saponin to LPS, the concentration of saponin is about about 5 μg per dose and the concentration of LPS is about 12.5 μg per dose. In some embodiments having this ratio of saponin to LPS, the concentration of saponin is about 6 μg per dose and the concentration of LPS is about 15 μg per dose. In some embodiments having this ratio of saponin to LPS, the concentration of saponin is about 7 μg per dose and the concentration of LPS is about 17.5 μg per dose. In some embodiments having this ratio of saponin to LPS, the concentration of saponin is about 8 μg per dose and the concentration of LPS is about 20 μg per dose. In some embodiments having this ratio of saponin to LPS, the concentration of saponin is about 9 μg per dose and the concentration of LPS is about 22.5 μg per dose. In some embodiments having this ratio of saponin to LPS, the concentration of saponin is about 10 μg per dose and the concentration of LPS is about 25 μg per dose.

### B. Sterols

Saponin presented in its quenched form with a sterol is effective in promoting T cell responses in human subjects. A sterol is a steroid alcohol and refers to any molecule having the 4-member ring structure characteristic of steroids and a hydroxyl (—OH) or ester (—OR) substitution at the 3-carbon position. Sterols are naturally present in the membranes of plants, animals, and microorganisms and are termed phytosterols, zoosterols, and mycosterols, respectively. A sterol may be further substituted at one or more of the other ring carbons, and may also contain various double bonds in the rings. Non-limiting examples of a sterol may include cholesterol, cholesteryl chloroformate, stigmasterol, sitosterol, ergosterol, lanosterol, desmosterol, or campesterol. Sterols generally associate with saponin to forms a stable, insoluble complex. In a specific embodiment of the composition described herein, the liposomal formulation comprises a saponin and, optionally, a LPS, where the saponin is complexed to a sterol. In an exemplary embodiment, the liposomal formulation comprises a saponin and, optionally, a LPS, where the saponin is complexed to cholesterol.

In certain embodiments of the liposomal formulation comprising a saponin complexed to a sterol, the ratio of saponin to sterol is from about 1:110 to 1:200. In some embodiments, the ratio of saponin to sterol is from about 1:110 to 1:150. In some preferred embodiments, the ratio of saponin to sterol is from about 1:120 to 1:150. In an exemplary embodiment, the ratio of saponin to sterol is about 1:125. Typically, the sterol acts to reduce the hemolytic activity of the saponin. In some aspects, the sterol acts to reduce the hemolytic activity of the saponin by 50%, 60%, 70%, 80%, 90% or even 100%.

The present disclosure also contemplates, in other preferred embodiments, a liposomal formulation comprising a saponin complexed to a sterol and a LPS, where the concentration of LPS is about 10 μg per dose or about 5 μg per dose. In certain embodiments, the saponin is complexed to a sterol, where the concentration of saponin is about 4 μg per dose or about 2 μg per dose. In an exemplary embodiment, the liposomal formulation comprises a saponin complexed to a sterol and a LPS, where the concentration of saponin is about 4 μg per dose and the concentration of LPS is about 10 μg per dose.

In another exemplary embodiment, the liposomal formulation comprises a saponin complexed to a sterol and a LPS, where the concentration of saponin is about 2 μg per dose and the concentration of LPS is about 5 μg per dose.

The present disclosure also contemplates, in other preferred embodiments, a liposomal formulation comprising a saponin complexed to a sterol and a LPS, where the saponin is an immunologically active saponin fraction derived from the bark of Quillaja saponaria Molina. In a preferred embodiment, the active saponin fraction is QS21. In other embodiments of the composition described herein, the saponin is synthetic. In an exemplary embodiment, the liposomal formulation comprises QS21 complexed to cholesterol and a LPS. In another exemplary embodiment, the liposomal formulation comprises a synthetic QS21 (SQS21) complexed to cholesterol and a LPS.

### C. Phospholipids

Liposomes have been employed for the delivery of subunit protein vaccine compositions and adjuvants. Liposomes are attractive delivery vehicles due to the ability to tailor the liposomal formulation to achieve desired lipid concentration, charge, size, and distribution or targeting of antigen and adjuvant. Numerous liposome-based systems have been evaluated including anionic, cationic, and neutral liposomes. It is contemplated herein that the lipid component of the liposomal formulation can comprise at least one of any lipid (which includes phospholipids) to form a stable liposome structure.

In certain embodiments of the composition provided herein, the liposomal formulation comprises at least one phospholipid. In some embodiments, the phospholipid is anionic. In some embodiments, the phospholipid is cationic. In other embodiments, the phospholipid has a neutral charge.

Table 1 provides a non-limiting list of exemplary lipids for use in the disclosure.

TABLE 1
Exemplary Lipids

| | |
|---|---|
| DLPC salt | 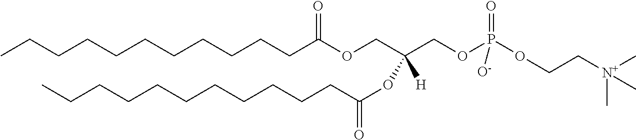 [SMILES: CCCCCCCCCCCC(=O)OC[C@H](COP(=O)([O-])OCC[N+](C)(C)C)OC(=O)CCCCCCCCCCC] |
| DMPC salt | 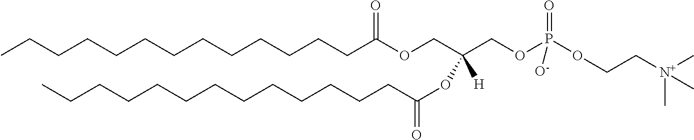 [SMILES: CCCCCCCCCCCCCC(=O)OC[C@H](COP(=O)([O-])OCC[N+](C)(C)C)OC(=O)CCCCCCCCCCCCC] |
| DPPC salt | 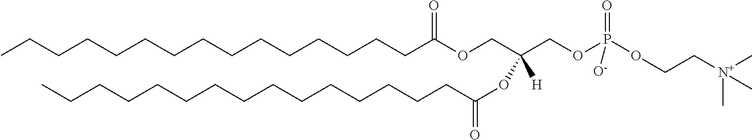 [SMILES: CCCCCCCCCCCCCCCC(=O)OC[C@H](COP(=O)([O-])OCC[N+](C)(C)C)OC(=O)CCCCCCCCCCCCCCC] |
| DSPC salt | 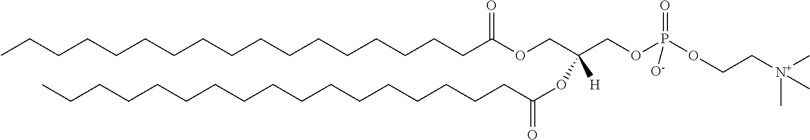 [SMILES: CCCCCCCCCCCCCCCCCC(=O)OC[C@H](COP(=O)([O-])OCC[N+](C)(C)C)OC(=O)CCCCCCCCCCCCCCCCC] |
| DOPC salt | 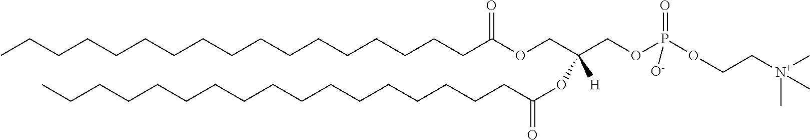 [SMILES: CCCCCCCCCCCCCCCCCC(=O)OC[C@H](COP(=O)([O-])OCC[N+](C)(C)C)OC(=O)CCCCCCCCCCCCCCCCC] |
| POPC salt | 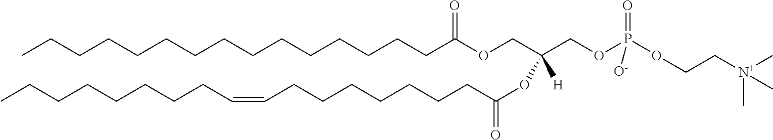 [SMILES: CCCCCCCC/C=C\CCCCCCCC(=O)O[C@H](COC(=O)CCCCCCCCCCCCCCC)COP(=O)([O-])OCC[N+](C)(C)C] |
| DLPG salt | 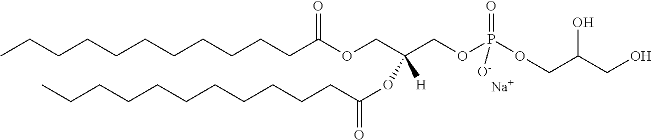 [SMILES: CCCCCCCCCCCC(=O)OC[C@H](COP(=O)([O-])OCC(O)CO)OC(=O)CCCCCCCCCCC ; [Na+]] |
| DMPG salt | 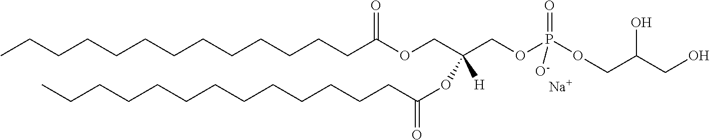 [SMILES: CCCCCCCCCCCCCC(=O)OC[C@H](COP(=O)([O-])OCC(O)CO)OC(=O)CCCCCCCCCCCCC ; [Na+]] |
| DPPG salt | 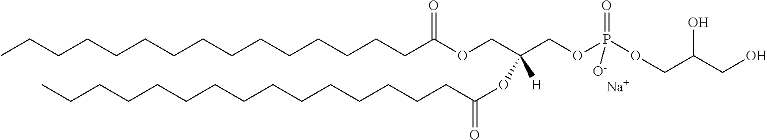 [SMILES: CCCCCCCCCCCCCCCC(=O)OC[C@H](COP(=O)([O-])OCC(O)CO)OC(=O)CCCCCCCCCCCCCCC ; [Na+]] |
| DSPG salt | 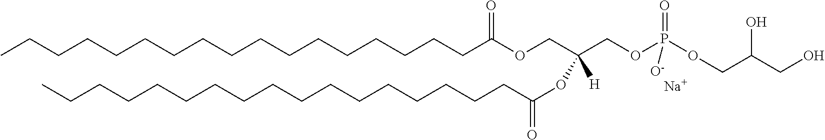 [SMILES: CCCCCCCCCCCCCCCCCC(=O)OC[C@H](COP(=O)([O-])OCC(O)CO)OC(=O)CCCCCCCCCCCCCCCCC ; [Na+]] |
| DOPG salt | 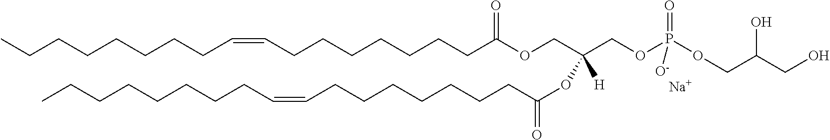 [SMILES: CCCCCCCC/C=C\CCCCCCCC(=O)OC[C@H](COP(=O)([O-])OCC(O)CO)OC(=O)CCCCCCC/C=C\CCCCCCCC ; [Na+]] |
| DSTAP salt | 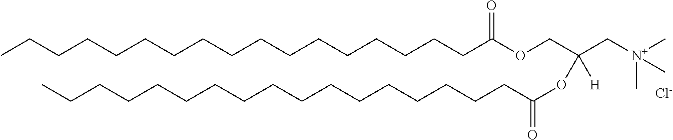 [SMILES: CCCCCCCCCCCCCCCCCC(=O)OCC(C[N+](C)(C)C)OC(=O)CCCCCCCCCCCCCCCCC ; [Cl-]] |
| DPTAP salt | 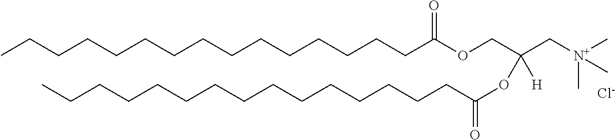 [SMILES: CCCCCCCCCCCCCCCC(=O)OCC(C[N+](C)(C)C)OC(=O)CCCCCCCCCCCCCCC ; [Cl-]] |
| DSPE salt | 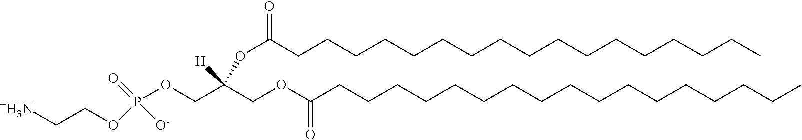 [SMILES: CCCCCCCCCCCCCCCCCC(=O)OC[C@H](COP(=O)([O-])OCC[NH3+])OC(=O)CCCCCCCCCCCCCCCCC] |
| DPPE salt | 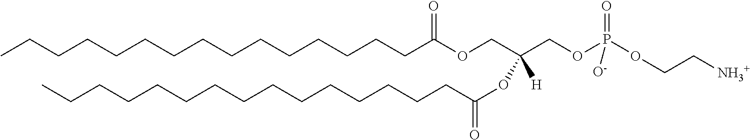 [SMILES: CCCCCCCCCCCCCCCC(=O)OC[C@H](COP(=O)([O-])OCC[NH3+])OC(=O)CCCCCCCCCCCCCCC] |
| DMPE salt | 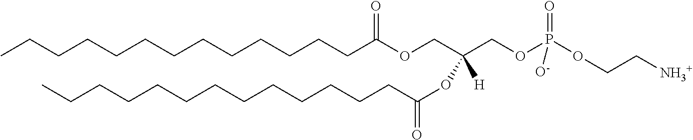 [SMILES: CCCCCCCCCCCCCC(=O)OC[C@H](COP(=O)([O-])OCC[NH3+])OC(=O)CCCCCCCCCCCCC] |
| DSPC salt | 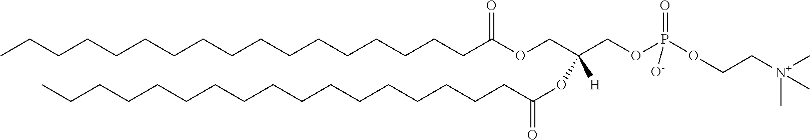 [SMILES: CCCCCCCCCCCCCCCCCC(=O)OC[C@H](COP(=O)([O-])OCC[N+](C)(C)C)OC(=O)CCCCCCCCCCCCCCCCC] |

In certain exemplary embodiments of the liposomal formulation described herein, the lipid component comprises at least one phospholipid selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, DLPE, DLPS 1,2-dilauroyl-sn-glycero-3-phospho-L-serine, DMPS 1,2-myristoyl-sn-glycero-3-phospho-L-serine, DPPS:1,2-dipalmitoyl-sn-glycero-3-phospho-L-serine, DSPS 1,2-distearoyl-sn-glycero-3-phospho-L-serine, DOPS 1,2-dioleoyl-sn-glycero-3-phospho-L-serine, POPS 1-palmitoyl-2-oleoyl-sn-glycero-3-phospho-L-serine, DLPI 1,2-dilauroyl-sn-glycero-3-phospho-(1′-myo-inositol), DMPI 1,2-myri stoyl-sn-glycero-3-phospho-(1′-myo-inositol), DPPI 1,2-dipalmitoyl-sn-glycero-3-phospho-(1′-myo-inositol), DSPI 1,2-distearoyl-sn-glycero-3-phosphoinositol, DOPI 1,2-dioleoyl-sn-glycero-3-phospho-(1′-myo-inositol), and POPI 1-palmitoyl-2-oleoyl-sn-glycero-3-phosphoinositol. It will be understood by the skilled practitioner that the phospholipids may be in salt form (e.g., ammonium or sodium salt).

### D. Liposome Characteristics

Size

The present disclosure provides liposomal formulations. The size of the liposomes provided herein can be assessed by known techniques in the art, including but not limited to, x-ray and laser diffraction, dynamic light scattering (DLS), CryoEM, or Malvern Zetasize. In some embodiments, the size of the liposome refers to the Z-average diameter.

The liposomes provided herein have an average diameter (i.e., the number average diameter) of 1 micrometer or less. It is particularly desirable that the average particle size (i.e., the number average diameter) of the liposome particles is about 900 nm or less, about 800 nm or less, about 700 nm or less, about 600 nm or less, about 500 nm or less, about 400 nm or less, 300 nm or less, or 200 nm or less, for example, from about 50 nm to about 900 nm, from about 50 nm to about 800 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, from about 50 nm to about 175 nm, from about 50 nm to about 150 nm, from about 50 nm to about 125 nm, from about 50 nm to about 100 nm.

The size of the liposomes described herein is typically about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, or is about 200 nm. It will be understood by the skilled practitioner that a liposome is made up of particles. The average particle size refers to the average diameter of the particles that make up the liposome.

An exemplary liposomal formulation of the present disclosure is capable of being filtered through at least a 0.45 micron filter. In an exemplary embodiment, the liposomal formulation is capable of being filtered through a 0.20 or 0.22 micron filter.

Volume

Certain embodiments of the present disclosure contemplate a liposomal formulation comprising a saponin and a LPS, where the formulation is in a volume suitable for use in a human dose. In some embodiments, the volume of the formulation is from about 0.5 ml to about 1.5 ml. In specific embodiments, the volume of the formulation is about 0.5 ml, about 0.6 ml, about 0.7 ml, about 0.8 ml, about 0.9 ml, about 1.0 ml, about 1.1 ml, about 1.2 ml, about 1.3 ml, about 1.4 ml, or about 1.5 ml. In certain embodiments, the volume of the formulation is from about 0.5 ml to about 0.75 ml, from about 0.75 ml to about 1.0 ml, from about 1.0 ml to about 1.25 ml, or from about 1.25 ml to about 1.5 ml.

Stability

The liposomal formulations provided herein are stable, allowing for ease of use, manufacturability, transportability, and storage. The physiochemical characteristics of the liposomal formulations, including, but not limited to liposomal size, is maintained over time, at various temperatures, and under various conditions.

The evolution of particle size over a function of time provides colloidal stability information. An exemplary stable liposomal formulations is one whose liposomes retain substantially the same z-average diameter size over a time period (e.g., a 30 day or 7 day time period) at different temperatures typically but not limited to 37, 25 or 5 degrees Celsius. By retaining substantially the same Z-average diameter size, it is meant that a liposome remains within 20%, 15%, 10%, 5%, of its original size over a 30 day time period. A particularly stable liposomal formulation is one whose particles retain substantially the same Z-average diameter size over a 30 day period at 25 degrees Celsius or even 37 degrees Celsius.

The stability of the liposomal formulation can be measured by techniques familiar to those of skill in the art. In some embodiments, the stability is observed visually. Visual inspection can include inspection for particulates, flocculence, or aggregates. Typically, colloidal stability is determined by the particle size of the lipsomes, such as by measuring the Z-average diameter and optionally expressed as change in size over time, or at various temperatures, or under certain conditions. In some embodiments, the stability is determined by assessing the increase in particle size. In some embodiments, stability is determined by measurement of the polydispersity index (PDI), for example with the use of the dynamic light scattering (DLS) technique. In other embodiments, stability is determined by measurement of the zeta potential with the use of the DLS technique.

In some embodiments, the Z-average diameter of the liposomes increases less than 50%, less than 40%, less than 30%, less than 25%, less than 20%, less than 15%, less than 12%, less than 10%, less than 7%, less than 5%, less than 3%, less than 1% over the time period assayed.

In some embodiments the polydispersity index of the liposomes is maintained at about 0.5, at about 0.4, at about 0.3, at about 0.2, at about 0.1 or at from about 0.1 to about 0.5, at about 0.1 to about 0.4, at from about 0.1 to about 0.3 or at about 0.1 to about 0.2.

### III. Exemplary Formulations

In one aspect, the liposomal formulation contains a saponin and, optionally, a LPS. In another aspect, the liposomal formulation contains a saponin complexed to a sterol and, optionally, a LPS. In another aspect, the liposomal formulation contains a saponin and a LPS, where the saponin is complexed to a sterol.

In certain embodiments the saponin is at a concentration of about 1 μg per dose to about 8 μg per dose and the LPS is at a concentration of about 3 μg per dose to about 20 μg per dose.

In one exemplary embodiment, the liposomal formulation comprises a saponin and, optionally, a LPS, where the saponin is complexed to a sterol at a ratio of about 1:110 to 1:200. In another exemplary embodiment, the liposomal formulation comprises a saponin and, optionally, a LPS, where the saponin is complexed to a sterol at a ratio of about 1:125.

In a specific embodiment the saponin is complexed to a sterol and the saponin is at a concentration of about 4 μg per dose. In a specific embodiment the saponin is complexed to a sterol and the saponin is at a concentration of about 2 μg per dose.

In a specific embodiment the saponin is complexed to a sterol and the LPS is present and at a concentration of about 10 μg per dose. In a specific embodiment the saponin is complexed to a sterol and the LPS is present at a concentration of about 5 μg per dose.

In an exemplary embodiment, the saponin is complexed to a sterol, the saponin is at a concentration of 4 μg per dose, and the LPS is present and is at a concentration of about 10 μg per dose. In another exemplary embodiment, the saponin is complexed to a sterol, the saponin is at a concentration of 2 μg per dose, and the LPS is present and is at a concentration of about 5 μg per dose.

In a preferred embodiment of the liposomal formulation described herein, the saponin is an immunologically active saponin fraction derived from the bark of Quillaja saponaria Molina. In an exemplary embodiment, the saponin fraction is QS21.

In specific embodiments, the saponin is synthetic. In certain embodiments, the liposomal formulation comprises synthetic QS21 (QS21) complexed to a sterol and, optionally, a LPS.

In an exemplary embodiment of the formulation provided herein, the saponin is complexed to cholesterol.

In an exemplary embodiment of the formulation provided herein, the formulation further comprises a phospholipid selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

In an exemplary embodiment, the LPS is GLA having the structure according to Formula (II) and where R¹, R³, R⁵ and R⁶ are C₁₁ alkyl; and R² and R⁴ are C₁₃ alkyl, or a pharmaceutically acceptable salt thereof. In another exemplary embodiment, the liposomal formulation comprises a saponin and a LPS, where the LPS is GLA having the structure according to Formula (II) and where R¹, R³, R⁵ and R⁶ are C₁₀ alkyl; and R² and R⁴ are C₈ alkyl, or a pharmaceutically acceptable salt thereof.

In still another exemplary embodiment, the LPS is MPL.

In a specific embodiment, the liposomal formulation is in a volume suitable for use in a human dose. In an exemplary embodiment, the volume of the liposomal formulation is from about 0.5 ml to about 1.5 ml.

In a specific exemplary embodiment, the liposomal formulation for administration to a human subject comprises a saponin and a LPS, where the saponin is at a concentration of about 1 μg per dose to about 10 μg per dose and the LPS is at a concentration of about 3 μg per dose to about 25 μg per dose, where the saponin is complexed to a sterol and the ratio of saponin to sterol is about 1:110 to about 1:200. In certain embodiments, the saponin is at a concentration of about 1 μg per dose to about 8 μg per dose and the LPS is at a concentration of about 3 μg per dose to about 20 μg per dose.

In an exemplary embodiment, the liposomal formulation for administration to a human subject comprises a saponin and a LPS, where the saponin is at a concentration of about 1 μg per dose to about 10 μg per dose and the LPS is at a concentration of about 3 μg per dose to about 25 μg per dose, where the saponin is complexed to a sterol and the ratio of saponin to sterol is about 1:125. In certain embodiments, the ratio of saponin to LPS is 1:2.5.

In an exemplary embodiment, the liposomal formulation comprises a saponin and a LPS, where the saponin is complexed to a sterol, the saponin is at a concentration of 4 μg per dose, the LPS is at a concentration of about 10 μg per dose, and where the saponin is complexed to a sterol and the ratio of saponin to sterol is about 1:125.

In another exemplary embodiment, the liposomal formulation comprises a saponin and a LPS, where the saponin is complexed to a sterol, the saponin is at a concentration of 2 μg per dose, the LPS is at a concentration of about 5 μg per dose, and where the saponin is complexed to a sterol and the ratio of saponin to sterol is about 1:125.

In certain exemplary embodiments, the liposomal formulation for administration to a human subject contains QS21 and a LPS, where the QS21 is at a concentration of about 1 μg per dose to about 10 μg per dose, the LPS is at a concentration of about 3 μg per dose to about 25 μg per dose, the QS21 is complexed to a sterol and the ratio of saponin to sterol is about 1:110 to about 1:200.

In certain exemplary embodiments, the liposomal formulation contains QS21 complexed to cholesterol in a ratio of about 1:110 to about 1:200 and a LPS. In a specific embodiment, the liposomal formulation comprising QS21 complexed to cholesterol in a ratio of about 1:110 to about 1:200 and a LPS further comprises a phospholipid selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

In certain exemplary embodiments, the liposomal formulation comprises QS21 complexed to cholesterol in a ratio of about 1:110 to about 1:200 and GLA according to Formula (II), where R¹, R³, R⁵ and R⁶ are C₁₁ alkyl; and R² and R⁴ are C₁₃ alkyl, or a pharmaceutically acceptable salt thereof.

In other exemplary embodiments, the liposomal formulation comprises QS21 complexed to cholesterol in a ratio of about 1:110 to about 1:200 and GLA according to Formula (II), where R¹, R³, R⁵ and R⁶ are C₁₀ alkyl; and R² and R⁴ are C₈ alkyl, or a pharmaceutically acceptable salt thereof.

In another exemplary embodiment, the liposomal formulation comprises QS21 complexed to cholesterol in a ratio of about 1:110 to about 1:200 and MPL.

In another exemplary embodiment, the liposomal formulation comprises saponin and, optionally, a lipopolysaccharide, wherein the saponin is complexed to a sterol and the weight ratio of saponin to sterol is about 1:110 to about 1:200, 1:110 to about 1:150, 1:120 to about 1:150, or about 1:125. The liposome formulation can comprise, e.g., a phospholipid and the weight ratio of phospholipid to sterol can be, for example, from 1:1 to about 10:1. In some aspects, the liposome formulation comprises a phospholipid and the weight ratio of phospholipid to sterol is about 4:1. The saponin can be, for example, at a concentration of about 0.5 μg per dose to about 10 μg per dose; at a concentration of about 1 μg per dose to about 10 μg per dose; at a concentration of about 1 μg per dose to about 8 μg per dose. The lipopolysaccharide is optionally present, when present, it can be, for example, at a concentration of 1.25 μg per dose to about 25 μg per dose, or at a concentration of about 3 μg per dose to about 25 μg per dose, although different dosage levels are contemplated. The saponin can be, for example at a concentration of about 1 μg per dose to about 8 μg per dose and the lipopolysaccharide can be at a concentration of about 3 μg per dose to about 20 μg per dose. The ratio of lipopolysaccharide to saponin can be, for example about 2.5 to 1. The saponin can be at a concentration, for example of about 4 μg per dose and the lipopolysaccharide can be, for example, at a concentration of about 10 μg per dose. The saponin can be at a concentration, for example of about 2 μg per dose and the lipopolysaccharide can be, for example, at a concentration of about 5 μg per dose. The formulations can comprise, for example, a saponin at a concentration of about 8 ug/ml, lipopolysaccharide at a concentration of about 20 ug/ml, phospholipid at a concentration of about 4 mg/ml, and sterol at a concentration of about 1 mg/ml. The formulations can be in a diluted form (e.g., 2 to 10 fold dilution or more) or a concentrated form (e.g. 2 to 10 fold concentration or more). In any of these embodiments, saponin can be an immunologically active saponin fraction derived from the bark of Quillaja saponaria Molina. The saponin can be, for example, QS21. In any of these embodiments, the sterol can be cholesterol although other sterols are contemplated. In any of these embodiments, the liposome can be made up of a phospholipid. Any suitable phospholipid can be used including, for example, DLPC, DMPC, DPPC, DSPC, DOPC, POPC, DLPG, DMPG, DPPG, DSPG, DOPG, DSTAP, DPTAP, DSPE, DPPE, DMPE, DLPE, DLPS, DMPS, DPPS, DSPS, DOPS, POPS, DLPI, DMPI, DPPI, DSPI, DOPI, or POPI. Any of the lipopolysaccharides described herein can be used as well as others known in the art. Immediate prior to administration, the formulation will be in a volume suitable for use in a human dose. Exemplary volumes include 0.5 ml to about 1.5 ml. An antigen can be mixed with the formulation. Any of the antigens described herein can be used as well as other suitable ones known in the art. The formulation can be used to elicit or enhance an immune response in a subject. The subject can be suffering from a number of diseases including, for example, cancer, an infectious disease, or an autoimmune disease. The subject can be human. Various amounts of saponin and LPS can be delivered per dose (e.g., 2 ug of saponin with 5 ug of LPS (e.g., GLA); 4 ug of saponin with 10 ug of LPS (e.g GLA).

Also provided is a method of manufacturing any of the saponin-containing liposomal formulations described herein comprising mixing the saponin with pre-formed sterol-containing liposomes. The saponin can be, for example, QS21 and, in some aspects, the crude saponin mixture Quil A is purified to obtain the saponin. In some aspects, the saponin is solubilized into buffer prior to mixing with liposomes. The pre-formed sterol-containing liposomes can be prepared by mixing the phospholipid and the sterol and reducing the particle size of the resultant liposomes via high pressure homogenization.

### IV. Pharmaceutical Compositions and Vaccine Compositions

In certain aspects, the liposomal formulations described herein are incorporated into pharmaceutical compositions or vaccine compositions. The polypeptides, antigens, polynucleotides, portions, variants, fusion polypeptides, etc., as described herein, may also be incorporated into pharmaceutical compositions or vaccine compositions. Pharmaceutical compositions generally comprise the liposomal formulations, in combination with a physiologically acceptable carrier. Vaccine compositions, also referred to as immunogenic compositions, generally comprise an antigens and one or more of the polypeptides, polynucleotides, portions, variants, fusion proteins, etc., as described herein.

In preferred embodiments, the pharmaceutical compositions contain the liposomal formulation provided herein and, optionally, an antigen. The liposomal formulations and the pharmaceutical compositions are optionally mixed with an antigen. In such embodiments, the liposomal formulations and the pharmaceutical compositions are formulated such that they are suitable for mixing with an antigen. In some preferred embodiments, the vaccine compositions contain the liposomal formulation provided herein and an antigen.

A. Antigen

An antigen may be any target epitope, molecule (including a biomolecule), molecular complex (including molecular complexes that contain biomolecules), subcellular assembly, cell or tissue against which elicitation or enhancement of immunoreactivity in a subject is desired. Frequently, the term antigen will refer to a polypeptide antigen of interest. However, antigen, as used herein, may also refer to a nucleic acid molecule (e.g., DNA or RNA) that encodes a polypeptide antigen. The antigen may also be a recombinant construct which encodes a polypeptide antigen of interest (e.g., an expression construct). Suitable antigens include, but are not limited to, a bacterial antigen, a viral antigen, a fungal antigen, a protozoan antigen, a plant antigen, a cancer antigen, or a combination thereto. The antigen described herein can be involved in, or derived from, for example, an infectious disease, cancer, autoimmune disease, allergy, asthma, or any other condition where stimulation of an antigen-specific immune response would be desirable or beneficial.

In certain embodiments the antigen may be derived from or is immunologically cross-reactive with at least one infectious pathogen that is associated with an infectious disease. In certain embodiments the antigen may be derived from or is immunologically cross-reactive with at least one epitope, biomolecule, cell, or tissue that is associated with cancer. In certain embodiments the antigen may be derived from or is immunologically cross-reactive with at least one epitope, biomolecule, cell, or tissue that is associated with an autoimmune disease.

It will be appreciated that the liposomal formulations and pharmaceutical compositions of the present invention can elicit an immune response in a human in instances where the compositions do not contain an antigen, In certain other embodiments the pharmaceutical compositions and vaccine compositions of the present disclosure contain an antigen or antigenic composition capable of eliciting an immune response in a human or other mammalian host. The antigen or antigenic composition may be capable of eliciting an immune response on its own or when combined with the formulations and compositions of the present invention. In some aspects, the formulations of the present invention enhance the ability of the antigen or antigenic composition to elicit an immune response in a human or other mammal.

The antigen or antigenic composition may include a composition derived from one or more bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans: H. ducreyi, Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, —B or —C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii, Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or other bacterial pathogens.

In certain embodiments the pharmaceutical compositions and vaccine compositions of the present disclosure contain an antigen or antigenic composition capable of eliciting an immune response in a human or other mammalian host in which the antigen or antigenic composition may include a composition derived from one or more infectious viruses such as from HIV-1, (such as tat, nef, gp120 or gp160), human herpes viruses, such as gD or derivatives thereof or Immediate Early protein such as ICP27 from HSV1 or HSV2, cytomegalovirus ((esp. Human) (such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, etc.), flaviviruses (e.g., Yellow Fever Virus, Dengue Virus, Tick-borne encephalitis virus, Japanese Encephalitis Virus) or Influenza virus (whole live or inactivated virus, split influenza virus, grown in eggs or MDCK cells, or whole flu virosomes (as described by Gluck, Vaccine, 1992, 10, 915-920) or purified or recombinant proteins thereof, such as HA, NP, NA, or M proteins, or combinations thereof).

In certain other embodiments the pharmaceutical compositions and vaccine compositions of the present disclosure contain an antigen or antigenic composition capable of eliciting an immune response in a human or other mammalian host in which the antigen or antigenic composition may include a composition derived from one or more parasites (See, e.g., John, D. T. and Petri, W. A., Markell and Voge's Medical Parasitology—9^{th} Ed., 2006, WB Saunders, Philadelphia; Bowman, D. D., Georgis' Parasitology for Veterinarians—8^{th} Ed., 2002, WB Saunders, Philadelphia) such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; or from a helminth capable of infecting a mammal, such as: (i) nematode infections (including, but not limited to, Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, and Strongyloides stercoralis); (ii) trematode infections (including, but not limited to, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica); and (iii) cestode infections (including, but not limited to, Taenia saginata and Taenia solium). Certain embodiments may therefore contemplate vaccine compositions that include an antigen derived from Schisostoma spp., Schistosoma mansonii, Schistosoma haematobium, and/or Schistosoma japonicum, or derived from yeast such as Candida spp., including C. albicans; Cryptococcus spp., including C. neoformans.

Certain preferred embodiments contemplate an antigen that is derived from at least one infectious pathogen such as a bacterium, a virus or a fungus, including an Actinobacterium such as M. tuberculosis or M. leprae or another mycobacterium; a bacterium such as a member of the genus Escherichia, Salmonella, Neisseria, Borrelia, Chlamydia, Clostridium or Bordetella; a virus such as a herpes simplex virus, a human immunodeficiency virus (HIV such as HIV-1 or HIV-2), an influenza virus, a parainfluenza virus, a measles virus, a mumps virus, a rubella virus, a coronavirus (such as SARS or MERS), a rotavirus, a norovirus, a picorna virus (such as a poliovirus, an enterovirus, or a coxsacchie virus), a veterinary pathogen, for example, a feline immunodeficiency virus (FIV), cytomegalovirus, Varicella Zoster Virus, hepatitis virus, Epstein Barr Virus (EBV), a flavivirus virus (such as dengue virus, Japanese encephalitis virus, yellow fever virus, Zika virus, Powassan virus or tick-borne encephalitis virus), a henipah virus (such as hendra or nipah virus), a bunyavirus (such as Hantavirus or Rift Valley Fever virus), an arenavirus (such as lassa virus, junin virus, machupo virus, or guanarito virus), a filovirus (such as Ebola virus or Marburg virus), a lyssavirus (such as Rabies virus), respiratory syncytial virus, human papilloma virus (HPV) and a cytomegalovirus; a fungus such as Aspergillus, Blastomyces, Coccidioides and Pneumocysti or a yeast, including Candida species such as C. albicans, C. glabrata, C. krusei, C. lusitaniae, C. tropicalis and C. parapsilosis; a parasite such as a protozoan, for example, a Plasmodium species including P. falciparum, P. vivax, P. malariae and P. ovale; or another parasite such as one or more of Acanthamoeba, Entamoeba histolytica, Angiostrongylus, Schistosoma mansonii, Schistosoma haematobium, Schistosoma japonicum, Cryptosporidium, Ancylostoma, Entamoeba histolytica, Entamoeba coli, Entamoeba dispar, Entamoeba hartmanni, Entamoeba polecki, Wuchereria bancrofti, Giardia, Toxoplasma gondii, and Leishmania. In specific embodiments, the antigen may be from, or related to antigens involved in tuberculosis, influenza, amebiasis, HIV, hepatitis, or Leishmaniasis.

According to the present disclosure, in certain aspects, the antigen included in the pharmaceutical compositions and vaccine compositions described herein is not derived from or associated with West Nile virus. In some aspects, the antigen is derived from or associated with TB, HIV, or malaria.

In some embodiments, the antigen is an influenza-related antigen. In some embodiments, the antigen is an influenza-causing antigen. In some embodiments, the antigen is from an influenza causing virus. In one embodiment, the antigen comprises hemagglutinin (HA) from H5N1. In one embodiment, the antigen comprises neuraminidase from H5N1.

For example, in certain embodiments, antigens are derived from Borrelia sp., the antigens may include nucleic acid, pathogen derived antigen or antigenic preparations, recombinantly produced protein or peptides, and chimeric fusion proteins. One such antigen is OspA. The OspA may be a full mature protein in a lipidated form by virtue of its biosynthesis in a host cell (Lipo-OspA) or may alternatively be a non-lipidated derivative. Such non-lipidated derivatives include the non-lipidated NS1-OspA fusion protein which has the first 81 N-terminal amino acids of the non-structural protein (NS1) of the influenza virus, and the complete OspA protein, and another, MDP-OspA is a non-lipidated form of OspA carrying 3 additional N-terminal amino acids.

Other specific antigens are derived from M. tuberculosis, for example Th Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, three, or four or more, polypeptides of M. tuberculosis are fused into a larger protein. Certain fusions include Ra12-TbH9-Ra35, Erd 14-DPV-MTI, DPV-MTI-MSL, Erd 14DPV-MTI-MSL-mTCC2, Erd14-DPV-MTI-MSL, DPV-MTI-MSL-mTCC2, TbH9-DPV-MTI (WO 99151748). Other antigens that may be used include antigens, combination of antigens, and fusion proteins described in US 2010/0129391, WO 2008/124647, and U.S. Pat. No. 8,486,414 incorporated herein by reference and for all purposes. In one exemplary embodiment, the fusion protein is ID93. In one exemplary embodiment, the fusion protein is ID91. In one exemplary embodiment, the fusion protein is ID97.

Other specific antigens are derived from Chlamydia, and include for example the High Molecular Weight Protein (HWMP) (WO 99/17741), ORF3 (EP 366 412), and putative membrane proteins (Pmps). Other Chlamydia antigens can be selected from the group described in WO 99128475. Certain antigens may be derived from Streptococcus spp, including S. pneumoniae (for example capsular polysaccharides and conjugates thereof, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial vaccine compositions comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Other specific antigens are derived from Hepatitis B. Derivatives of Hepatitis B Surface antigen are well known in the art and include, inter alia, those PreS1, PreS2, S antigens set forth described in European Patent applications EP-A414 374; EP-A-0304 578, and EP 198474.

In other embodiments, the antigen is derived from the Human Papilloma Virus (HPV) considered to be responsible for genital warts (HPV 6 or HPV 11 and others), and the HPV viruses responsible for cervical cancer (HPV16, HPV18 and others). Particular antigens include L1 particles or capsomers, and fusion proteins comprising one or more antigens selected from the HPV 6 and HPV 11 proteins E6, E7, L1, and L2. Certain forms of fusion protein include L2E7 as disclosed in WO 96/26277, and protein D(1/3)-E7 disclosed in GB 9717953.5 (PCT/EP98/05285). Additional possible antigens include HPV 16, 18, 33, 58 antigens. For example, L1 or L2 antigen monomers, or L1 or L2 antigens presented together as a virus like particle (VLP) or the L1 alone protein presented alone in a VLP or caposmer structure. Such antigens, virus like particles and capsomer are per se known. See for example WO94/00152, WO94/20137, WO94/05792, and WO93/02184.

In other embodiments, the antigen is a fusion protein. Fusion proteins may be included alone or as fusion proteins such as E7, E2 or F5 for example; particular embodiments include a VLP comprising L1E7 fusion proteins (WO 96/11272). Particular HPV 16 antigens comprise the early proteins E6 or F7 in fusion with a protein D carrier to form Protein D-E6 or E7 fusions from HPV 16, or combinations thereof; or combinations of E6 or E7 with L2 (WO 96/26277). Alternatively the HPV 16 or 18 early proteins E6 and E7, may be presented in a single molecule, for example a Protein D-E6/E7 fusion. Compositions may optionally contain either or both E6 and E7 proteins front HPV 18, for example in the form of a Protein D-E6 or Protein D-E7 fusion protein or Protein D E6/E7 fusion protein. Compositions may additionally comprise antigens from other HPV strains, for example from strains HPV 31 or 33.

Antigens may also be derived from parasites that cause Malaria. For example, antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No. PCT/EP92/02591, published as WO 93/10152 claiming priority from UK patent application No. 9124390.7. When expressed in yeast RTS is produced as a lipoprotein particle, and when it is co-expressed with the S antigen from HBV it produces a mixed particle known as RTS,S.

TRAP antigens are described in the International Patent Application No. PCT/GB89/00895 published as WO 90/01496. An embodiment of the present disclosure is a Malaria vaccine where the antigenic preparation comprises a combination of the RTS,S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27125, Pfs16, Pfs48/45, Pfs230 and their analogues in Plasmodium spp.

In one embodiment, the antigen is derived from a cancer cell, as may be useful for the immunotherapeutic treatment of cancers. For example, the antigen may be a tumor rejection antigen such as those for prostate, breast, colorectal, lung, pancreatic, renal or melanoma cancers. Exemplary cancer or cancer cell-derived antigens include MAGE 1, 3 and MAGE 4 or other MAGE antigens such as those disclosed in WO99/40188, PRAME, BAGE, Lage (also known as NY Eos 1) SAGE and HAGE (WO 99/53061) or GAGE (Robbins and Kawakami, 1996 Current Opinions in Immunology 8, pps 628-636; Van den Eynde et al., International Journal of Clinical & Laboratory Research (1997 & 1998); Correale et al. (1997), Journal of the National Cancer Institute 89, p. 293. These non-limiting examples of cancer antigens are expressed in a wide range of tumor types such as melanoma, lung carcinoma, sarcoma and bladder carcinoma. See, e.g., U.S. Pat. No. 6,544,518.

Other tumor-specific antigens are include, but are not restricted to, tumor-specific or tumor-associated gangliosides such as GM₂, and GM₃ or conjugates thereof to carrier proteins; or a self peptide hormone such as whole length Gonadotrophin hormone releasing hormone (GnRH, WO 95/20600), a short 10 amino acid long peptide, useful in the treatment of many cancers. In another embodiment prostate antigens are used, such as Prostate specific antigen (PSA), PAP, PSCA (e.g., Proc. Nat. Acad. Sci. USA 95(4) 1735-1740 1998), PSMA or, in one embodiment an antigen known as Prostase. (e.g., Nelson, et al., Proc. Natl. Acad. Sci. USA (1999) 96: 3114-3119; Ferguson, et al. Proc. Natl. Acad. Sci. USA 1999. 96, 3114-3119; WO 98/12302; U.S. Pat. No. 5,955,306; WO 98/20117; U.S. Pat. Nos. 5,840,871 and 5,786,148; WO 00/04149. Other prostate specific antigens are known from WO 98/137418, and WO/004149. Another is STEAP (PNAS 96 14523 14528 7-12 1999).

Other tumor associated antigens useful in the context of the present disclosure include: Plu-1 (J Biol. Chem 274 (22) 15633-15645, 1999), HASH-1, HasH-2, Cripto (Salomon et al Bioessays 199, 21:61-70, U.S. Pat. No. 5,654,140) and Criptin (U.S. Pat. No. 5,981,215). Additionally, antigens particularly relevant for vaccine compositions in the therapy of cancer also comprise tyrosinase and survivin.

The herein disclosed embodiments may also comprise a cancer antigen that will be useful against any cancer characterized by tumor associated antigen expression, such as HER-2/neu expression or other cancer-specific or cancer-associated antigens.

Diagnosis of cancer in a subject having or suspected of being at risk for having cancer may be accomplished by any of a wide range of art-accepted methodologies, which may vary depending on a variety of factors including clinical presentation, degree of progression of the cancer, the type of cancer, and other factors. Examples of cancer diagnostics include histopathological, histocytochemical, immunohistocytochemical and immunohistopathological examination of patient samples (e.g., blood, skin biopsy, other tissue biopsy, surgical specimens, etc.), PCR tests for defined genetic (e.g., nucleic acid) markers, serological tests for circulating cancer-associated antigens or cells bearing such antigens, or for antibodies of defined specificity, or other methodologies with which those skilled in the art will be familiar. See, e.g., U.S. Pat. Nos. 6,734,172; 6,770,445; 6,893,820; 6,979,730; 7,060,802; 7,030,232; 6,933,123; 6,682,901; 6,587,792; 6,512,102; 7,078,180; 7,070,931; JP5-328975; Waslylyk et al., 1993 Eur. J. Bioch. 211(7):18.

Liposomal formulations, pharmaceutical compositions and vaccine compositions and methods according to certain embodiments of the present disclosure may also be used for the prophylaxis or therapy of autoimmune diseases, which include diseases, conditions or disorders where a host's or subject's immune system detrimentally mediates an immune response that is directed against “self” tissues, cells, biomolecules (e.g., peptides, polypeptides, proteins, glycoproteins, lipoproteins, proteolipids, lipids, glycolipids, nucleic acids such as RNA and DNA, oligosaccharides, polysaccharides, proteoglycans, glycosaminoglycans, or the like, and other molecular components of the subjects cells and tissues) or epitopes (e.g., specific immunologically defined recognition structures such as those recognized by an antibody variable region complementarity determining region (CDR) or by a T cell receptor CDR.

Autoimmune diseases are thus characterized by an abnormal immune response involving either cells or antibodies, that are in either case directed against normal autologous tissues. Autoimmune diseases in mammals can generally be classified in one of two different categories: cell-mediated disease (i.e., T-cell) or antibody-mediated disorders. Non-limiting examples of cell-mediated autoimmune diseases include multiple sclerosis, rheumatoid arthritis, Hashimoto thyroiditis, type I diabetes mellitus (Juvenile onset diabetes) and autoimmune uvoretinitis. Antibody-mediated autoimmune disorders include, but are not limited to, myasthenia gravis, systemic lupus erythematosus (or SLE), Graves' disease, autoimmune hemolytic anemia, autoimmune thrombocytopenia, autoimmune asthma, cryoglobulinemia, thrombic thrombocytopenic purpura, primary biliary sclerosis and pernicious anemia. The antigen(s) associated with: systemic lupus erythematosus is small nuclear ribonucleic acid proteins (snRNP); Graves' disease is the thyrotropin receptor, thyroglobulin and other components of thyroid epithelial cells (Akamizu et al., 1996; Kellerman et al., 1995; Raju et al., 1997; and Texier et al., 1992); pemphigus is cadherin-like pemphigus antigens such as desmoglein 3 and other adhesion molecules (Memar et al., 1996: Stanley, 1995; Plott et al., 1994; and Hashimoto, 1993); and thrombic thrombocytopenic purpura is antigens of platelets. (See, e.g., U.S. Pat. No. 6,929,796; Gorski et al. (Eds.), Autoimmunity, 2001, Kluwer Academic Publishers, Norwell, M A; Radbruch and Lipsky, P. E. (Eds.) Current Concepts in Autoimmunity and Chronic Inflammation (Curr. Top. Microbiol. and Immunol.) 2001, Springer, N.Y.)

In certain embodiments, the compositions of the disclosure will be particularly applicable in treatment of the elderly and/or the immunosuppressed, including subjects on kidney dialysis, subjects on chemo-therapy and/or radiation therapy, transplant recipients, and the like. Such individuals generally exhibit diminished immune responses to vaccine compositions and therefore use of the compositions of the disclosure can enhance the immune responses achieved in these subjects.

In other embodiments, the antigen or antigens used in the compositions of the disclosure include antigens associated with respiratory diseases, such as those caused or exacerbated by bacterial infection (e.g. pneumococcal), for the prophylaxis and therapy of conditions such as chronic obstructive pulmonary disease (COPD). COPD is defined physiologically by the presence of irreversible or partially reversible airway obstruction in patients with chronic bronchitis and/or emphysema (Am J Respir Crit. Care Med. 1995 November; 152(5 Pt 2):S77-121). Exacerbations of COPD are often caused by bacterial (e.g. pneumococcal) infection (Clin Microbiol Rev. 2001 April; 14(2):336-63).

In a preferred embodiment, the liposomal formulation is contained within a pharmaceutical composition. In another preferred embodiment, the liposomal formulation is contained within a vaccine composition. In an exemplary embodiment, the pharmaceutical composition comprises the liposomal formulation and an antigen. In another exemplary embodiment, the vaccine composition comprises the liposomal formulation and an antigen. In some such exemplary embodiments, the antigen is associated with an infectious disease, cancer, or an autoimmune disease. In an exemplary embodiment, the liposomal formulations and pharmaceutical formulations can be used to treat diseases such as infectious disease, cancer, or an autoimmune disease. In an exemplary embodiment, the liposomal formulations and pharmaceutical formulations can be used to elicit enhanced immune responses in mammals, including humans, having diseases such as infectious disease, cancer, or an autoimmune disease. In such embodiments, the liposomal formulations and pharmaceutical formulations may or may not further comprise an antigen and/or nucleic acid encoding an antigen.

According to certain embodiments disclosed herein, the pharmaceutical composition and vaccine composition may, in lieu of comprising an antigen, comprise a nucleic acid encoding an antigen. For example, in embodiments, the pharmaceutical composition and vaccine composition may contain at least one recombinant expression construct which comprises a promoter operably linked to a nucleic acid sequence encoding an antigen. In certain further embodiments the recombinant expression construct is present in a viral vector, such as an adenovirus, adeno-associated virus, herpesvirus, lentivirus, poxvirus or retrovirus vector. Compositions and methods for making and using such expression constructs and vectors are known in the art, for the expression of polypeptide antigens as provided herein, for example, according to Ausubel et al. (Eds.), Current Protocols in Molecular Biology, 2006 John Wiley & Sons, NY. Non-limiting examples of recombinant expression constructs generally can be found, for instance, in U.S. Pat. Nos. 6,844,192; 7,037,712; 7,052,904; 7,001,770; 6,106,824; 5,693,531; 6,613,892; 6,875,610; 7,067,310; 6,218,186; 6,783,981; 7,052,904; 6,783,981; 6,734,172; 6,713,068; 5,795,577 and 6,770,445 and elsewhere, with teachings that can be adapted to the expression of polypeptide antigens as provided herein, for use in certain presently disclosed embodiments.

The compositions provided herein may comprise at least one additional immunostimulant in addition to the saponin and optional lipopolysaccharide which typically act as immunostimulants in the formulations and compositions of the present invention. An immunostimulant is any substance that enhances or potentiates an immune response (antibody and/or cell-mediated) to an antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995).

For example, and by way of background (see, e.g., U.S. Pat. No. 6,544,518) immunostimulatory oligonucleotides containing ummethylated CpG dinucleotides (“CpG”) are known as being adjuvants when administered by both systemic and mucosal routes (WO 96/02555, EP 468520, Davis et al., J. Immunol, 1998. 160(2):870-876; McCluskie and Davis, J. Immunol., 1998, 161(9):4463-6). CpG is an abbreviation for cytosine-guanosine dinucleotide motifs present in DNA. The central role of the CG motif in immunostimulation was elucidated by Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; where the dinucleotide CG motif is not methylated, but other unmethylated CpG sequences are known to be immunostimulatory and may be used in certain embodiments of the present disclosure. CpG when formulated into vaccine compositions, may be administered in free solution together with free antigen (WO 96/02555; McCluskie and Davis, supra) or covalently conjugated to an antigen (PCT Publication No. WO 98/16247), or formulated with a carrier such as aluminium hydroxide (e.g., Davis et al. supra, Brazolot-Millan et al., Proc. NatL Acad. Sci., USA, 1998, 95(26), 15553-8).

Other illustrative oligonucleotides for use in compositions of the present disclosure will often contain two or more dinucleotide CpG motifs separated by at least three, more preferably at least six or more nucleotides. The oligonucleotides of the present disclosure are typically deoxynucleotides. In one embodiment the internucleotide in the oligonucleotide is phosphorodithioate, or more preferably a phosphorothioate bond, although phosphodiester and other internucleotide bonds are within the scope of the disclosure including oligonucleotides with mixed internucleotide linkages. Methods for producing phosphorothioate oligonucleotides or phosphorodithioate are described in U.S. Pat. Nos. 5,666,153, 5,278,302 and WO95/26204.

Other examples of oligonucleotides have sequences that are disclosed in the following publications; for certain herein disclosed embodiments the sequences preferably contain phosphorothioate modified internucleotide linkages:

CPG 7909: Cooper et al., “CPG 7909 adjuvant improves hepatitis B virus vaccine seroprotection in antiretroviral-treated HIV-infected adults.” AIDS, 2005 Sep. 23; 19(14):1473-9.

CpG 10101: Bayes et al., “Gateways to clinical trials.” Methods Find. Exp. Clin. Pharmacol. 2005 April; 27(3):193-219. Vollmer J., “Progress in drug development of immunostimula-tory CpG oligodeoxynucleotide ligands for TLR9.” Expert Opinion on Biological Therapy. 2005 May; 5(5): 673-682.

Alternative CpG oligonucleotides may comprise variants of the preferred sequences described in the above-cited publications that differ in that they have inconsequential nucleotide sequence substitutions, insertions, deletions and/or additions thereto. The CpG oligonucleotides utilized in certain embodiments of the present disclosure may be synthesized by any method known in the art (e.g., EP 468520). Conveniently, such oligonucleotides may be synthesized utilizing an automated synthesizer. The oligonucleotides are typically deoxynucleotides. In a preferred embodiment the internucleotide bond in the oligonucleotide is phosphorodithioate, or more preferably phosphorothioate bond, although phosphodiesters are also within the scope of the presently contemplated embodiments. Oligonucleotides comprising different internucleotide linkages are also contemplated, e.g., mixed phosphorothioate phophodiesters. Other internucleotide bonds which stabilize the oligonucleotide may also be used.

B. Carriers and Excipients

The pharmaceutical compositions and vaccine compositions of the disclosure may be formulated using any of a variety of well-known procedures. In certain embodiments, the pharmaceutical compositions and vaccine compositions are prepared as stable emulsions (e.g., oil-in-water emulsions) or as aqueous solutions.

In certain applications, the compositions disclosed herein may be delivered via oral administration to a subject. As such, these compositions may be formulated with an inert diluent or with an assailable edible carrier, or they may be enclosed in hard- or soft-shell gelatin capsule, or they may be compressed into tablets, or they may be incorporated directly with the food of the diet.

In certain circumstances it will be desirable to deliver the compositions disclosed herein parenterally, subcutaneously, intravenously, intradermally, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical composition forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxy groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective for treatment of leprosy. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known to one of ordinary skill in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an unacceptable allergic or similar untoward reaction when administered to a human. The preparation of an aqueous composition that contains a protein as an active ingredient is well understood to one of ordinary skill in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions of the present disclosure may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

A pharmaceutical composition or vaccine composition may, alternatively, contain an immunostimulant and a DNA molecule encoding one or more of the polypeptides or fusion polypeptides as described above, such that a desired polypeptide is generated in situ. In such compositions, the DNA encoding the fusion protein may be present within any of a variety of delivery systems known to those of ordinary skill in the art, including nucleic acid expression systems, bacterial and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a particular embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells.

C. Kits and Articles of Manufacture

Also contemplated in certain embodiments are kits containing the herein described liposomal formulations, pharmaceutical compositions and vaccine compositions, which may be provided in one or more containers. In one embodiment, all components of the liposomal formulation are present together in a single container. In certain embodiments, all components of the pharmaceutical compositions are present together in a single container. In certain embodiments, all components of the vaccine compositions are present together in a single container. In other embodiments, components of the pharmaceutical compositions and vaccine compositions may be in two or more containers. In a preferred embodiment, the liposomal formulation is provided in one container, and the antigen is provided in another container.

The kits of the disclosure may further comprise instructions for use as herein described or instructions for mixing the materials contained in the vials. In some embodiments, the material in the vial is dry or lyophilized. In some embodiments, the material in the vial is liquid.

A container according to such kit embodiments may be any suitable container, vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Typically such a container may be made of a material that is compatible with the intended use and from which recovery of the contained contents can be readily achieved. Non-limiting examples of such containers include glass and/or plastic sealed or re-sealable tubes and ampules, including those having a rubber septum or other sealing means that is compatible with withdrawal of the contents using a needle and syringe. Such containers may, for instance, by made of glass or a chemically compatible plastic or resin, which may be made of, or may be coated with, a material that permits efficient recovery of material from the container and/or protects the material from, e.g., degradative conditions such as ultraviolet light or temperature extremes, or from the introduction of unwanted contaminants including microbial contaminants. The containers are preferably sterile or sterilizeable, and made of materials that will be compatible with any carrier, excipient, solvent, vehicle or the like, such as may be used to suspend or dissolve the herein described vaccine compositions and/or immunological adjuvant compositions and/or antigens and/or recombinant expression constructs, etc.

### V. Methods of Making the Composition of the Disclosure

The present inventors have advantageously discovered that the saponin containing liposomes can be made in a process whereby the saponin (and optional LPS) is mixed with pre-formed liposomes to create the formulations described herein.

As provided herein, one method of making exemplary liposomal formulations involves mixing the LPS with DOPC and cholesterol in a 4 to 1 phospholipid to cholesterol ratio by weight. The mixing step is performed in a round-bottomed glass flask in the presence of chloroform before evaporating the chloroform under vacuum and hydrating the thin film with phosphate buffer. In certain embodiments, the LPS is SLA. In an exemplary embodiment, the LPS is GLA. In some embodiments, a further step includes water bath sonication (for the 10-ml scale) or high-pressure homogenization (for the ≥100 ml scale) to uniformly reduce the particle size to nanometer (nm) dimensions (70-130 nm average particle size based on DLS measurements). The high pressure homogenization may be conducted using the Microfluidics 110EH or 110P microfluidizer models at 20,000 psi, 10-15° C., and 5 homogenization passes.

In certain embodiments, QS21 is obtained via HPLC purification of the crude saponin mixture Quil-A. In a preferred embodiment, QS21 may be separately solubilized into phosphate buffer and then mixed into the prepared liposomes containing LPS and cholesterol prior to sterile filtration. In an exemplary embodiment, the final manufactured product is filter-sterilized with a 0.22-μm filter (Millipore Steripak GP10) and contains 4 mg/mL DOPC, 1 mg/mL cholesterol, 20 ug/ml GLA, and 8 mg/ml QS21. A pharmaceutical composition or vaccine composition may be prepared by mixing the liposomal formulation with an antigen in a 1 to 1 ratio prior to administration. Following manufacture, the formulation may stored at 5° C. and placed on a stability monitoring program that includes measurement of particle size (via DLS) and visual appearance at time of manufacture and 1 week, 2 weeks, 1 month, 3 months, 6 months, 12 months, and so forth after the date of manufacture. In addition, LPS and QS21 concentrations may be measured by HPLC with charged aerosol detection (CAD) at time of manufacture and at 6 months, 12 months, and so forth after the date of manufacture. In some embodiments, liposomal formulations described herein are stored at higher temperatures (25° C., 37° C., and 60° C.) for accelerated stability monitoring.

### VI. Methods of Eliciting or Enhancing an Immune Response

Provided herein are methods of eliciting or enhancing an immune response in a subject, including the step of administering to a subject in need thereof a liposomal formulation, a pharmaceutical composition or a vaccine composition described herein. In some embodiments, the formulations or compositions further comprise an antigen where the antigen is a polypetide antigen or a nucleic acid molecule encoding a polypeptide antigen. In some such embodiments, the formulations or compositions are suitable for mixing with a polypetide antigen or a nucleic acid molecule encoding a polypeptide antigen

In the embodiments provided herein, the subject is a mammal (e.g., an animal including farm animals (cows, pigs, goats, horses, etc.), pets (cats, dogs, etc.), and rodents (rats, mice, etc.), or a human. In one embodiment, the subject is a human. In another embodiment, the subject is a non-human mammal. In another embodiment, the non-human mammal is a dog, cow, or horse.

In exemplary embodiments, the liposomal formulations disclosed herein are incorporated into vaccine compositions. The liposomal formulations described herein can be used for eliciting or enhancing an immune response in the subject (including a non-specific response and an antigen-specific response). In some embodiments, the immune response comprises a systemic immune response. In some embodiments, the immune response comprises a mucosal immune response. Elicitation or enhancement of an immune response includes stimulating an immune response, and boosting an immune response.

The disclosure thus provides compositions for altering (i.e., increasing or decreasing in a statistically significant manner, for example, relative to an appropriate control as will be familiar to persons skilled in the art) immune responses in a host capable of mounting an immune response. As will be known to persons having ordinary skill in the art, an immune response may be any active alteration of the immune status of a host, which may include any alteration in the structure or function of one or more tissues, organs, cells or molecules that participate in maintenance and/or regulation of host immune status. Typically, immune responses may be detected by any of a variety of well known parameters, including but not limited to in vivo or in vitro determination of: soluble immunoglobulins or antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death); or any other criterion by which the presence of an immune response may be detected. Accordingly, the formulations can act to enhance and/or induce antibody production, (e.g., induce production of neutralizing antibodies; enhance antigen specific antibody responses).

Immune responses may often be regarded, for instance, as discrimination between self and non-self structures by the cells and tissues of a host's immune system at the molecular and cellular levels, but the disclosure should not be so limited. For example, immune responses may also include immune system state changes that result from immune recognition of self molecules, cells or tissues, as may accompany any number of normal conditions such as typical regulation of immune system components, or as may be present in pathological conditions such as the inappropriate autoimmune responses observed in autoimmune and degenerative diseases. As another example, in addition to induction by up-regulation of particular immune system activities (such as antibody and/or cytokine production, or activation of cell mediated immunity) immune responses may also include suppression, attenuation or any other down-regulation of detectable immunity, which may be the consequence of the antigen selected, the route of antigen administration, specific tolerance induction or other factors.

Determination of the induction of an immune response by the vaccine compositions of the present disclosure may be established by any of a number of well known immunological assays with which those having ordinary skill in the art will be readily familiar. Such assays include, but need not be limited to, to in vivo or in vitro determination of: soluble antibodies; soluble mediators such as cytokines, lymphokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are widely known and may be found, for example in Lefkovits (Immunology Methods Manual: The Comprehensive Sourcebook of Techniques, 1998; see also Current Protocols in Immunology; see also, e.g., Weir, Handbook of Experimental Immunology, 1986 Blackwell Scientific, Boston, Mass.; Mishell and Shigii (eds.) Selected Methods in Cellular Immunology, 1979 Freeman Publishing, San Francisco, Calif.; Green and Reed, 1998 Science 281:1309 and references cited therein).

Detection of the proliferation of antigen-reactive T cells may be accomplished by a variety of known techniques. For example, T cell proliferation can be detected by measuring the rate of DNA synthesis, and antigen specificity can be determined by controlling the stimuli (such as, for example, a specific desired antigen- or a control antigen-pulsed antigen presenting cells) to which candidate antigen-reactive T cells are exposed. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a particular antigen may be quantified.

Detection of antigen-specific antibody production may be achieved, for example, by assaying a sample (e.g., an immunoglobulin containing sample such as serum, plasma or blood) from a host treated with a vaccine according to the present disclosure using in vitro methodologies such as radioimmunoassay (RIA), enzyme linked immunosorbent assays (ELISA), equilibrium dialysis or solid phase immunoblotting including Western blotting. In preferred embodiments ELISA assays may further include antigen-capture immobilization of the target antigen with a solid phase monoclonal antibody specific for the antigen, for example, to enhance the sensitivity of the assay. Elaboration of soluble mediators (e.g., cytokines, chemokines, lymphokines, prostaglandins, etc.) may also be readily determined by enzyme-linked immunosorbent assay (ELISA), for example, using methods, apparatus and reagents that are readily available from commercial sources (e.g., Sigma, St. Louis, Mo.; see also R & D Systems 2006 Catalog, R & D Systems, Minneapolis, Minn.).

Another way of assessing the immunogenicity of the pharmaceutical compositions or vaccine compositions disclosed herein where the nucleic acid molecule encodes a protein antigen is to express the recombinant protein antigen for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within protein antigens.

Any number of other immunological parameters may be monitored using routine assays that are well known in the art. These may include, for example, antibody dependent cell-mediated cytotoxicity (ADCC) assays, secondary in vitro antibody responses, flow immunocytofluorimetric analysis of various peripheral blood or lymphoid mononuclear cell subpopulations using well established marker antigen systems, immunohistochemistry or other relevant assays. These and other assays may be found, for example, in Rose et al. (Eds.), Manual of Clinical Laboratory Immunology, 5^{th} Ed., 1997 American Society of Microbiology, Washington, D.C.

Accordingly it is contemplated that the vaccine compositions provided herein will be capable of eliciting or enhancing in a host at least one immune response that is selected from a T_{H}1-type T lymphocyte response, a T_{H}2-type T lymphocyte response, a cytotoxic T lymphocyte (CTL) response, an antibody response, a cytokine response, a lymphokine response, a chemokine response, and an inflammatory response. In certain embodiments the immune response may comprise at least one of production of one or a plurality of cytokines where the cytokine is selected from interferon-gamma (IFN-γ), tumor necrosis factor-alpha (TNF-α), production of one or a plurality of interleukins where the interleukin is selected from IL-1, IL-2, IL-3, IL-4, IL-6, IL-8, IL-10, IL-12, IL-13, IL-16, IL-18 and IL-23, production one or a plurality of chemokines where the chemokine is selected from MIP-1α, MIP-1β, RANTES, CCL4 and CCL5, and a lymphocyte response that is selected from a memory T cell response, a memory B cell response, an effector T cell response, a cytotoxic T cell response and an effector B cell response. See, e.g., WO 94/00153; WO 95/17209; WO 96/02555; U.S. Pat. Nos. 6,692,752; 7,084,256; 6,977,073; 6,749,856; 6,733,763; 6,797,276; 6,752,995; 6,057,427; 6,472,515; 6,309,847; 6,969,704; 6,120,769; 5,993,800; 5,595,888; Smith et al., 1987 J Biol Chem. 262:6951; Kriegler et al., 1988 Cell 53:45 53; Beutler et al., 1986 Nature 320:584; U.S. Pat. Nos. 6,991,791; 6,654,462; 6,375,944.

The efficacy of the compositions provided herein can also be determined in vivo by challenging appropriate animal models with the pathogen of interest infection.

The compositions described herein may be used to enhance protective immunity against one or more bacterial pathogens such as Neisseria spp, including N. gonorrhea and N. meningitidis (for example capsular polysaccharides and conjugates thereof, transferrin-binding proteins, lactoferrin binding proteins, PilC, adhesins); S. pyogenes (for example M proteins or fragments thereof, C5A protease, lipoteichoic acids), S. agalactiae, S. mutans: H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, —B or —C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or other bacterial pathogens.

The compositions described herein may be used to enhance protective immunity against a virus. Such viruses and viral antigens include, for example, HIV-1, (such as tat, nef, gp120 or gp160), human herpes viruses (such as gD or derivatives thereof or Immediate Early protein such as ICP27 from HSV1 or HSV2), cytomegalovirus ((esp. Human, such as gB or derivatives thereof), Rotavirus (including live-attenuated viruses), Epstein Barr virus (such as gp350 or derivatives thereof), Varicella Zoster Virus (such as gpl, II and IE63), or from a hepatitis virus such as hepatitis B virus (for example Hepatitis B Surface antigen or a derivative thereof), hepatitis A virus, hepatitis C virus and hepatitis E virus, or from other viral pathogens, such as paramyxoviruses: Respiratory Syncytial virus (such as F and G proteins or derivatives thereof), parainfluenza virus, measles virus, mumps virus, human papilloma viruses (for example HPV6, 11, 16, 18, etc.), flaviviruses (e.g., dengue virus, Japanese encephalitis virus, yellow fever virus, Zika virus, Poswanan virus, tick-borne encephalitis virus) or Influenza virus (whole live or inactivated virus, split influenza virus, grown in eggs or MDCK cells, or whole flu virosomes (as described by Gluck, Vaccine, 1992, 10, 915-920) or purified or recombinant proteins thereof, such as HA, NP, NA, or M proteins, or combinations thereof). According to the present disclosure, the compositions described herein do not elicit or enhance protective immunity against West Nile virus.

The compositions described herein may be used to enhance protective immunity against one or more parasites (See, e.g., John, D. T. and Petri, W. A., Markell and Voge's Medical Parasitology—9^{th} Ed., 2006, WB Saunders, Philadelphia; Bowman, D. D., Georgis' Parasitology for Veterinarians—8^{th} Ed., 2002, WB Saunders, Philadelphia) such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; or from a helminth capable of infecting a mammal, such as: (i) nematode infections (including, but not limited to, Enterobius vermicularis, Ascaris lumbricoides, Trichuris trichiura, Necator americanus, Ancylostoma duodenale, Wuchereria bancrofti, Brugia malayi, Onchocerca volvulus, Dracanculus medinensis, Trichinella spiralis, and Strongyloides stercoralis); (ii) trematode infections (including, but not limited to, Schistosoma mansoni, Schistosoma haematobium, Schistosoma japonicum, Schistosoma mekongi, Opisthorchis sinensis, Paragonimus sp, Fasciola hepatica, Fasciola magna, Fasciola gigantica); and (iii) cestode infections (including, but not limited to, Taenia saginata and Taenia solium). In certain embodiments, the antigen is derived from Schisostoma spp., Schistosoma mansonii, Schistosoma haematobium, and/or Schistosoma japonicum, or derived from yeast such as Candida spp., including C. albicans; Cryptococcus spp., including C. neoformans. infectious pathogen such as a bacterium, a virus or a fungus, including an Actinobacterium such as M. tuberculosis or M leprae or another mycobacterium; a bacterium such as a member of the genus Salmonella, Neisseria, Borrelia, Chlamydia or Bordetella; a virus such as a herpes simplex virus, a human immunodeficiency virus (HIV), a feline immunodeficiency virus (FIV), cytomegalovirus, Varicella Zoster Virus, hepatitis virus, Epstein Barr Virus (EBV), Zika virus (ZIKV) respiratory syncytial virus, human papilloma virus (HPV) and a cytomegalovirus; HIV such as HIV-1 or HIV-2; a fungus such as Aspergillus, Blastomyces, Coccidioides and Pneumocysti or a yeast, including Candida species such as C. albicans, C. glabrata, C. krusei, C. lusitaniae, C. tropicalis and C. parapsilosis; a parasite such as a protozoan, for example, a Plasmodium species including P. falciparum, P. vivax, P. malariae and P. ovale; or another parasite such as one or more of Acanthamoeba, Entamoeba histolytica, Angiostrongylus, Schistosoma mansonii, Schistosoma haematobium, Schistosoma japonicum, Cryptosporidium, Ancylostoma, Entamoeba histolytica, Entamoeba coli, Entamoeba dispar, Entamoeba hartmanni, Entamoeba polecki, Wuchereria bancrofti, Giardia, and Leishmania.

The compositions described herein may be used to enhance protective immunity against at least one antigen derived from cancer, including adenocarcinoma, choroidal melanoma, acute leukemia, acoustic neurinoma, ampullary carcinoma, anal carcinoma, astrocytoma, basal cell carcinoma, pancreatic cancer, bladder cancer, bronchial carcinoma, non-small cell lung cancer (NSCLC), breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, urethral cancer, urologic tumors, urothelial carcinoma, vulva cancer, and cervical carcinoma.

The compositions described herein may be used to enhance protective immunity against one or more antigens derived from autoimmune diseases, such as multiple sclerosis, rheumatoid arthritis, Hashimoto thyroiditis, type I diabetes mellitus (Juvenile onset diabetes) and autoimmune uvoretinitis. Antibody-mediated autoimmune disorders include, but are not limited to, myasthenia gravis, systemic lupus erythematosus (or SLE), Graves' disease, autoimmune hemolytic anemia, autoimmune thrombocytopenia, autoimmune asthma, cryoglobulinemia, thrombic thrombocytopenic purpura, primary biliary sclerosis and pernicious anemia.

Typical routes of administration of the liposomal formulation, pharmaceutical composition, and vaccine composition include, without limitation, oral, topical, parenteral, sublingual, buccal, rectal, vaginal, intravenous, intradermal, transdermal, intranasal, intramucosal, or subcutaneous. In some exemplary embodiments, administration of the liposomal formulation, pharmaceutical composition, and vaccine composition is intramuscular, ocular, parenteral, or pulmonary.

In preferred embodiments, the method of administering the liposomal formulation described herein, the pharmaceutical composition described herein, and the vaccine composition described herein elicits or enhances an immune response in a subject.

In preferred embodiments, the method of administering the liposomal formulation described herein, the pharmaceutical composition described herein, and the vaccine composition described herein elicits or enhances an immune response in a subject afflicted with cancer, an infectious disease, or an autoimmune disease.

In exemplary embodiments, the method of administering the liposomal formulation described herein, the pharmaceutical composition described herein, and the vaccine composition described herein elicits or enhances an immune response in a human subject afflicted with cancer, an infectious disease, or an autoimmune disease.

It will also be understood that the methods of treatment of the present disclosure may include the administration of the compositions of the disclosure either alone or in conjunction with other agents and, as such, the therapeutic vaccine may be one of a plurality of treatment components as part of a broader therapeutic treatment regime.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

### EXAMPLES

### Example 1

Exemplary method for synthesis of the liposomal formulation GLA-LSQ (e.g., GLA* or SLA as the LPS), Q21 as the saponin, cholesterol as the sterol, and dioleoyl phosphatidylcholine as the phospholipid and having a saponin to sterol weight ratio of 1:125 of the GLA*-LSQ formulation or SLA-LSQ formulation.

To manufacture an exemplary GLA*-LSQ or SLA-LSQ liposomal formulation, GLA* or SLA is first mixed with dioleoyl phosphatidylcholine and cholesterol (4:1 phospholipid:cholesterol w:w ratio) in chloroform in a glass round-bottomed flask before evaporating the chloroform under vacuum and hydrating the thin film with phosphate buffer. Water bath sonication (for the 10-ml scale) or high-pressure homogenization (for the ≥100 ml scale) uniformly reduces the particle size to nanodimensions (70-130 nm average size based on dynamic light scattering [DLS]). The high pressure homogenization is conducted using the Microfluidics 110EH or 110P microfluidizer models at 20,000 psi, 10-15° C., and 5 homogenization passes. The QS21 molecule is obtained via HPLC purification of the crude saponin mixture Quil A. QS21 is separately solubilized into phosphate buffer and then mixed into the prepared SLA- or GLA*-liposomes prior to sterile filtration. The process is currently reproducible and robust. An exemplary final manufactured product is filter-sterilized with a 0.22-μm filter (Millipore Steripak GP10) and contains 4 mg/ml DOPC, 1 mg/ml cholesterol, 20 ug/ml GLA* or SLA, and 8 ug/ml QS21, currently designed for 1:1 mixing with antigen prior to administration. Following manufacture, the formulation is stored at 5° C. and placed on a stability monitoring program including measurement of particle size (via DLS) and visual appearance at time of manufacture and 1 week, 2 weeks, 1 month, 3 months, 6 months, 12 months, etc. after the date of manufacture. In addition, GLA* or SLA and QS21 concentrations are measured by HPLC with charged aerosol detection (CAD) at time of manufacture and at 6 months, 12 months, and so forth after the date of manufacture. In batches manufactured at IDRI, particle size and adjuvant concentration monitoring indicate good stability for the SLA-LSQ and GLA*-LSQ formulations. Samples are also stored at higher temperatures (25° C., 37° C., and 60° C.) for accelerated stability monitoring.

### Example 2

Comparison of ID93+GLA-SE and ID93+GLA-LSQ as a boost in BCG-primed guinea pigs.

The goal of this study is to determine an optimal adjuvant formulation for use with the ID93 vaccine in BCG primed guinea pigs. The ID93 vaccine is a recombinant subunit vaccine antigen formulated as a fusion protein from 4 Mtb proteins associated with virulence and latency (Rv2608, Rv3619, Rv3620 and Rv1813). The final 891 amino acid fusion protein has a predicted mass of 93 KDa. ID93 was tested in combination with two different adjuvant formulations, GLA*-SE and GLA*-LSQ and the protective effficacy of the vaccine in BCG-prime guinea pigs was determined. 80 female guinea pigs were used for the study primed intradermally with BCG and rested for 3 months. Immunization with the ID93 vaccine was 3 times, 3 weeks apart (days 0, 21, and 42). Challenge with low dose aerosol (1.17×10⁷ cfu/ml) M. tuberculosis Beijing 4619, 10 wks after the 3^{rd} immunization. ID93 dose was 10 ug. Adjuvant A was GLA*-LSQ with 5 ug GLA* and 2 ug QS21. Adjuvant B was GLA-SE(5 ug GLA). Group 1 was the only group not primed with BCG and was administered saline alone, group 2 was administered saline, group 3 was administered adjuvant A, group 4 was administered ID93 and adjuvant A, and group 5 was administered ID93 and adjuvant B.

At 60 days following infection, the ID93-GLA-SE vaccine had significantly reduced bacterial load in the lung and spleen compared to the saline control, similar to the BCG-prime group. In addition, the ID93-GLA-SE group had decreased bacteria in the mediastinal lymph node compared to the saline group, whereas the reduction of bacteria in the BCG-prime group was not statistically significant at this time point. The only group to show improved survival compared to the BCG-prime group was ID93+GLA-LSQ. ID93+GLA-LSQ had significantly reduced bacterial in the spleen at both 30 and 60 days following infection compared to the saline group, but no significant reduction of bacteria in the lung or MDL.

### Example 3

A Phase 1, Randomized, Double Blind Clinical Trial to Evaluate the Safety, Tolerability, and Immunogenicity of the Vaccine Candidates ID93+GLA*-LSQ and ID93+GLA-SE Administered Intramuscularly in Healthy Adult Subjects

A randomized, double blind clinical trial is underway to evaluate the safety, tolerability and immunogenicity of the ID93 recombinant protein antigen alone or formulated with GLA-SE or GLA*-LSQ adjuvant in 70 healthy adults 18-49 years of age. The four treatment groups are outlined in Table 1 below. Subjects received a total of 3 doses administered intramuscularly on Days 1, 29 and 57. Subjects will be monitored for approximately 422 days (365 days following the third study injection), including safety laboratory analyses done just prior to and 7 days following each study injection. Blood samples will be obtained for immunological assays (Luminex, intracellular cytokine staining at Days 1 and 71, and antibody analysis at Days 1 and 85).

TABLE 1

| | | | Timing of |
|---|---|---|---|
| Group | N | Study Injections | Study Injections |
| 1 | 20 | 10 μg ID93 + 5 μg GLA*-LSQ | Days 1, 29, 57 |
| 2 | 20 | 10 μg ID93 + 10 μg GLA*-LSQ | Days 1, 29, 57 |
| 3 | 20 | 10 μg ID93 + 5 μg GLA-SE | Days 1, 29, 57 |
| 4 | 10 | 10 μg ID93 | Days 1, 29, 57 |

Glucopyranosyl Lipid A (GLA*) is a synthetic Toll-like Receptor 4 (TLR4) agonist. GLA is formulated in a stable oil-in-water emulsion (SE) to yield the adjuvant formulation GLA-SE. Due to the TLR4 activity of the GLA molecule, the combination of GLA-SE with a recombinant protein antigen (ID93) results in a Th1-type T cell response. GLA*-LSQ is a liposomal formulation that includes GLA and the saponin QS-21. GLA formulated with liposomes has been shown to stimulate a robust immune response, but the addition of additional immunostimulatory ligands such as QS-21 increase the Th1 immune responses (Christensen D et al., Expert Rev Vaccines 2011; 10:513-21). QS-21 is derived from the soap bark tree (Quillaja Saponaria) and has been shown to elicit both CD4 T cells that express IFNγ and TNF and produce cytotoxic T lymphocytes against numerous antigens.

GLA* is formulated in a liposomal composition with QS-21 (LSQ) to generate the adjuvant GLA*-LSQ and is supplied as 20 μg/mL GLA* combined with 8 μg/mL QS-21 in single use vials. GLA*-LSQ appears as a hazy liquid. Each 2 mL vial contains a fill volume of 0.4 mL and must be stored at 2-8° C. The following are directions regarding injection reconstitution procedures: Group 1: 10 μg ID93+5 μg GLA*-LSQ: Reconstitute a vial of ID93 by adding 1.25 mL of WFI as described above (concentration: 80 μg/mL ID93). Add 0.2 mL of the reconstituted ID93 and 0.2 mL of WFI to a 0.4 mL vial of GLA*-LSQ and mix thoroughly. The total volume in this final admixed vial is now 0.8 mL (concentrations: 20 μg/mL ID93; 10 μg/mL GLA). Draw>0.5 mL of the mixed preparation into a 1 mL syringe and replace the needle with a 23-25-gauge 1-1½-inch needle for IM injection. Remove any air bubbles and prime the syringe to deliver 0.5 mL (10 μg ID93 and 5 μg GLA). Adhere to standard hospital policies for syringe and dose preparation to ensure that the required dose is administered. Group 2: 10 μg ID93+10 μg GLA*-LSQ: Reconstitute a vial of ID93 by adding 1.25 mL of WFI as described above (concentration: 80 μg/mL ID93). Add 0.15 mL of the reconstituted ID93, 0.45 mL of WFI, and 0.2 mL of GLA*-LSQ to a separate 0.4 mL vial of GLA*-LSQ and mix thoroughly. The total volume in this final admixed vial is now 1.2 mL (concentrations: 10 μg/mL ID93; 10 μg/mL GLA). Draw>1.0 mL of the mixed preparation into a 2.5 or 3 mL syringe and replace the needle with a 23-25-gauge 1-1½-inch needle for IM injection. Remove any air bubbles and prime the syringe to deliver 1.0 mL (10 μg ID93 and 10 μg GLA*). Adhere to standard hospital policies for syringe and dose preparation to ensure that the required dose is administered. Group 3: 10 μg ID93+5 μg GLA*-SE: Reconstitute a vial of ID93 by adding 1.25 mL of WFI as described above (concentration: 80 μg/mL ID93). Add 0.2 mL of the reconstituted ID93 and 0.2 mL of WFI to a 0.4 mL vial of GLA-SE and mix thoroughly. The total volume in this final admixed vial is now 0.8 mL (concentrations: 20 μg/mL ID93; 10 μg/mL GLA). Draw>0.5 mL of the mixed preparation into a 1 mL syringe and replace the needle with a 23-25-gauge 1-1½-inch needle for IM injection. Remove any air bubbles and prime the syringe to deliver 0.5 mL (10 μg ID93 and 5 μg GLA*). Adhere to standard hospital policies for syringe and dose preparation to ensure that the required dose is administered. Group 4: 10 μg ID93 alone: Reconstitute a vial of ID93 by adding 1.25 mL of WFI as described above (concentration: 80 μg/mL ID93). Add 0.3 mL of the reconstituted ID93 and 0.9 mL WFI to a sterile empty vial and mix thoroughly. The total volume in this final admixed vial is now 1.2 mL (concentration: 20 μg/mL ID93). Draw>0.5 mL of the mixed preparation into a 1 mL syringe and replace the needle with a 23-25-gauge 1-1½-inch needle for IM injection. Remove any air bubbles and prime the syringe to deliver 0.5 mL (10 μg ID93). Adhere to standard hospital policies for syringe and dose preparation to ensure that the required dose is administered.

Antibody responses, measured by IgG antibody responses to ID93, will be summarized by study Day (1 and 85) using descriptive statistics. Changes from baseline to each visit will be presented. A graph of immunological response data over time for each dose will be presented with confidence limits. Response rates for IgG and cytokines will be presented with exact confidence intervals and compared between treatment groups using Fisher's exact test. The magnitude of cytokine concentrations will be compared using analysis of variance when data are normally distributed or the appropriate non-parametric analytic method in the event that the data distribution is non-Gaussian.

We claim:

1. A liposomal formulation for administration to a human subject comprising a saponin and a lipopolysaccharide, wherein the saponin is at a concentration of about 1 μg per dose to about 10 μg per dose and the lipopolysaccharide is at a concentration of about 3 μg per dose to about 25 μg per dose, wherein the weight ratio of lipopolysaccharide to saponin is about 2.5 to 1.

2. The formulation of claim 1, wherein the saponin is at a concentration of about 1 μg per dose to about 8 μg per dose and the lipopolysaccharide is at a concentration of about 3 μg per dose to about 20 μg per dose.

3. The formulation of claim 1 or claim 2, wherein the saponin is complexed to a sterol.

4. The formulation of claim 3, wherein the weight ratio of saponin to sterol is about 1:110 to about 1:200.

5. The formulation of claim 3, wherein the weight ratio of saponin to sterol is about 1:110 to about 1:150.

6. The formulation of claim 3, wherein the weight ratio of saponin to sterol is about 1:120 to about 1:150.

7. The formulation of claim 3, wherein the weight ratio of saponin to sterol is about 1:125.

8. The formulation of any one of the above claims, wherein the saponin is at a concentration of about 4 μg per dose.

9. The formulation of any one the above of claims, wherein the lipopolysaccharide is at a concentration of about 10 μg per dose.

10. The formulation of any one of claims 1 to 7, wherein the saponin is at a concentration of about 2 μg per dose.

11. The formulation of any one of claims 1 to 7 or claim 10, wherein the lipopolysaccharide is at a concentration of 5 μg per dose.

12. The formulation of any one of the above claims, wherein the saponin is an immunologically active saponin fraction derived from the bark of Quillaja saponaria Molina.

13. The formulation of claim 12, wherein the saponin fraction is QS21.

14. The formulation of any one of claims 1 to 11, wherein the saponin is synthetic.

15. The formulation of any one of claims 3 to 14, wherein the sterol is cholesterol.

16. The formulation of any one of the above claims, further comprising a phospholipid.

17. The formulation of claim 16, wherein the phospholipid is selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

18. The formulation of any one of claims 1 to 17, wherein the lipopolysaccharide is glucopyranosyl lipid A (GLA).

19. The formulation of any one of claims 1 to 17, wherein the lipopolysaccharide has the following formula: 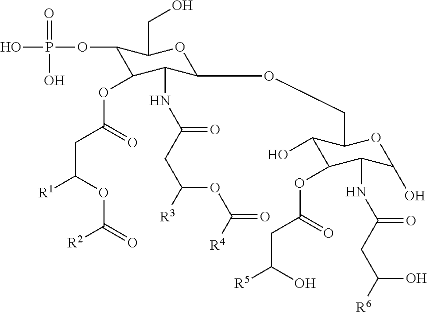
wherein R¹, R³, R⁵ and R⁶ are C₁₁ alkyl; and R² and R⁴ are C₁₃ alkyl, or a pharmaceutically acceptable salt thereof.

20. The formulation of any one of claims 1 to 17 wherein the lipopolysaccharide has the formula: 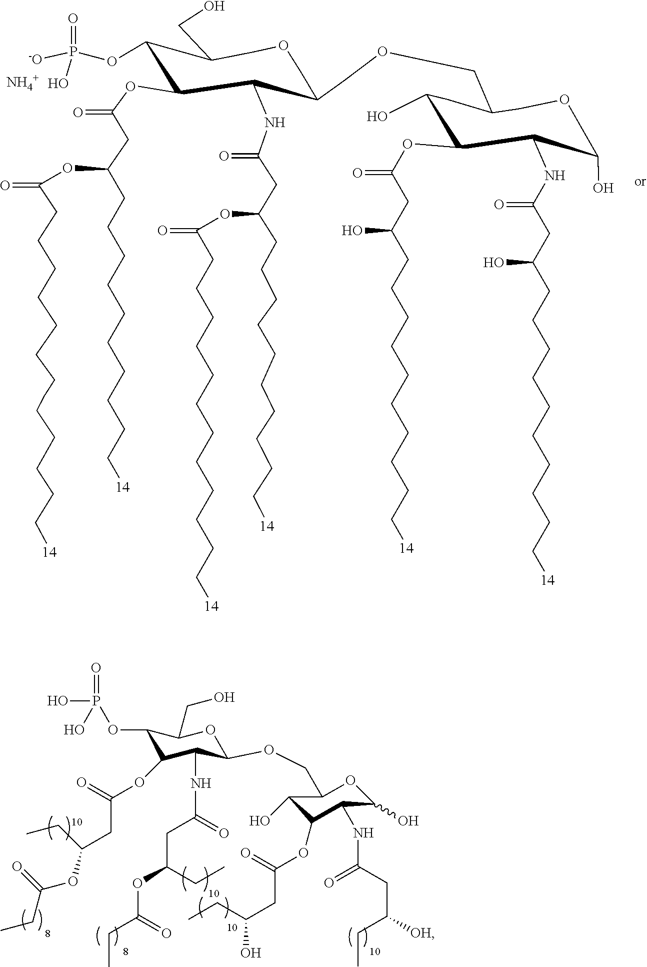
or a pharmaceutically acceptable salt thereof.

21. The formulation of any one of claims 1 to 17, wherein the lipopolysaccharide has the following formula: 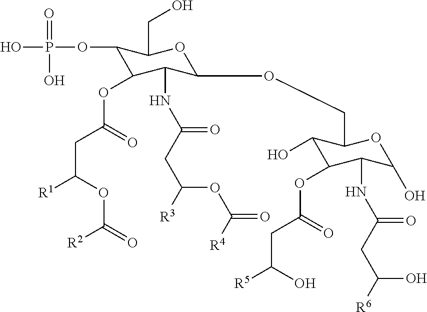
wherein R¹, R³, R⁵ and R⁶ are C10 alkyl; and R² and R⁴ are C8 alkyl, or a pharmaceutically acceptable salt thereof.

22. The formulation of any one of claims 1 to 17, wherein the lipopolysaccharide is monophosphoryl lipid A (MPL).

23. The formulation of any one of claims 1 to 22, wherein the formulation is in a volume suitable for use in a human dose.

24. The formulation of claim 23, wherein the volume is from about 0.5 ml to about 1.5 ml.

25. A pharmaceutical composition comprising any one of the formulations of claims 1 to 24.

26. The pharmaceutical composition of claim 25, further comprising an antigen.

27. A vaccine composition comprising any one of the formulations of claims 1 to 24 and an antigen.

28. The composition of claim 26 or 27, wherein the antigen is derived from or is immunologically cross-reactive with (i) at least one infectious pathogen that is associated with an infectious disease, (ii) at least one epitope, biomolecule, cell, or tissue that is associated with cancer, or (iii) at least one epitope, biomolecule, cell, or tissue that is associated with an autoimmune disease, thereby eliciting or enhancing an immune response.

29. A method of eliciting or enhancing an immune response in a subject, the method comprising administering to the subject a liposomal formulation of any one of claims 1 through 24, a pharmaceutical composition of claim 25 or 26, or a vaccine composition of claim 27 or 28.

30. The method of claim 29 wherein the liposomal formulation of any one of claims 1 through 24, a pharmaceutical composition of claim 25 or 26, or a vaccine composition of claim 27 or 28 is administered in combination with an antigen.

31. The method of claim 29 or claim 30, wherein the subject is afflicted with cancer, an infectious disease, or an autoimmune disease.

32. The method of any one of claims 29-31, wherein the subject is a human.

33. A liposomal formulation for administration to a human subject comprising a saponin and, optionally, a lipopolysaccharide, wherein the saponin is complexed to a sterol and the weight ratio of saponin to sterol is about 1:110 to about 1:200.

34. The formulation of claim 33 wherein the liposome formulation comprises a phospholipid and the weight ratio of phospholipid to sterol is from 1:1 to about 10:1

35. The formulation of claim 33 wherein the liposome formulation comprises a phospholipid and the weight ratio of phospholipid to sterol is about 4:1.

36. The formulation of claim 33 wherein the saponin is at a concentration of about 0.5 μg per dose to about 10 μg per dose.

37. The formulation of claim 33 wherein the saponin is at a concentration of about 1 μg per dose to about 10 μg per dose.

38. The formulation of claim 33 wherein the saponin is at a concentration of about 1 μg per dose to about 8 μg per dose.

39. The formulation of any one of claims 33 to 38 wherein the lipopolysaccharide is present and is at a concentration of 1.25 μg per dose to about 25 μg per dose.

40. The formulation of any one claims 33 to 39 wherein the lipopolysaccharide is present and is at a concentration of about 3 μg per dose to about 25 μg per dose

41. The formulation of claim 33, wherein the saponin is at a concentration of about 1 μg per dose to about 8 μg per dose and the lipopolysaccharide is present and at a concentration of about 3 μg per dose to about 20 μg per dose.

42. The formulation of any one of claims 33 to 41, wherein the formulation comprises a saponin at a concentration of about 8 ug/ml, lipopolysaccharide at a concentration of about 20 ug/ml, phospholipid at a concentration of about 4 mg/ml, and sterol at a concentration of about 1 mg/ml.

43. The formulation of claim 42 in a diluted form, preferably a 2 to 10 fold dilution.

44. The formulation of claim 42 in a concentrated form, preferably a 2 to 10 fold concentration.

45. The formulation of any one of claims 33 to 44, wherein the ratio of saponin to sterol is about 1:110 to about 1:150.

46. The formulation of any one of claims 33 to 44, wherein the ratio of saponin to sterol is about 1:120 to about 1:150.

47. The formulation of any one of claims 33 to 44, wherein the ratio of saponin to sterol is about 1:125.

48. The formulation of any one of claims 33 to 47, wherein the ratio of lipopolysaccharide to saponin is about 2.5 to 1.

49. The formulation of any one of claims 33 to 48, wherein the saponin is at a concentration of about 4 μg per dose.

50. The formulation of any one of claims 33 to 49, wherein the lipopolysaccharide is at a concentration of about 10 μg per dose.

51. The formulation of any one of claims 33 to 48, wherein the saponin is at a concentration of about 2 μg per dose.

52. The formulation of any one of claims 33 to 48 or claim 51, wherein the lipopolysaccharide is at a concentration of 5 μg per dose.

53. The formulation of any one of claims 33 to 52, wherein the saponin is an immunologically active saponin fraction derived from the bark of Quillaja saponaria Molina

54. The formulation of claim 53, wherein the saponin fraction is QS21.

55. The formulation of any one of claims 33 to 54, wherein the sterol is cholesterol.

56. The formulation of any one of claims 33 to 55, comprising a phospholipid wherein the phospholipid is selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

57. The formulation of claim 56, wherein the phospholipid is DOPC.

58. The formulation of any one of claims 33 to 57, wherein the lipopolysaccharide is glucopyranosyl lipid A (GLA).

59. The formulation of any one of claims 33 to 57, wherein the lipopolysaccharide has the following formula: 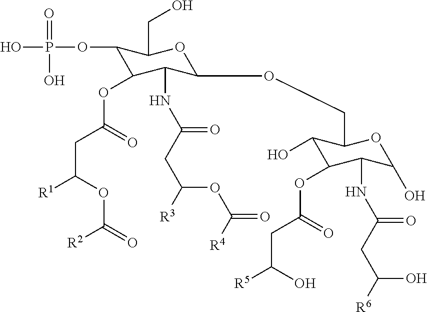
wherein R¹, R³, R⁵ and R⁶ are C11 alkyl; and R² and R⁴ are c13 alkyl, or a pharmaceutically acceptable salt thereof.

60. The formulation of claims 33 to 57 wherein the lipopolysaccharide has the following formula: 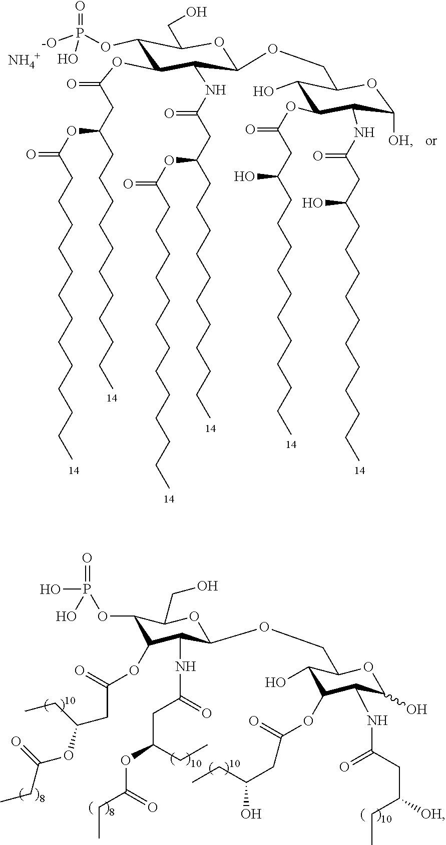
or a pharmaceutically acceptable salt thereof.

61. The formulation of any one of claims 33 to 57, wherein the lipopolysaccharide has the following formula: 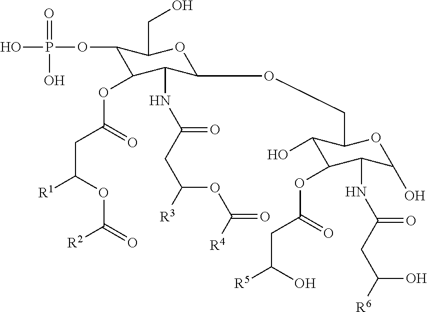
wherein R¹, R³, R⁵ and R⁶ are C₁₀ alkyl; and R² and R⁴ are C₈ alkyl, or a pharmaceutically acceptable salt thereof.

62. The formulation of any one of claims 33 to 57, wherein the lipopolysaccharide is monophosphoryl lipid A (MPL).

63. The formulation of any one of claims 33 to 43 and 45 to 62, wherein the formulation is in a volume suitable for use in a human dose.

64. The formulation of claim 63, wherein the volume is from about 0.5 ml to about 1.5 ml.

65. A pharmaceutical composition comprising any one of the formulations of claims 33 to 43 and 45 to 62.

66. The pharmaceutical composition of claim 65, further comprising an antigen.

67. A vaccine composition comprising any one of the formulations of claims 33 to 43 and 45 to 62 and an antigen.

68. The composition of claim 66 or 67, wherein the antigen is derived from or is immunologically cross-reactive with (i) at least one infectious pathogen that is associated with an infectious disease, (ii) at least one epitope, biomolecule, cell, or tissue that is associated with cancer, or (iii) at least one epitope, biomolecule, cell, or tissue that is associated with an autoimmune disease, thereby eliciting or enhancing an immune response.

69. A method of eliciting or enhancing an immune response in a subject, the method comprising administering to the subject a liposomal formulation of any one of claims 33 to 43 and 45 to 62, a pharmaceutical composition of claim 65 or 66, or a vaccine composition of claim 67 or 68.

70. The method of claim 69 wherein the liposomal formulation of any one of claims 33 to 43 and 45 to 62, a pharmaceutical composition of claim 65 or 66, or a vaccine composition of claim 67 or 68 is administered in combination with an antigen.

71. The method of claim 69 or claim 70, wherein the subject is afflicted with cancer, an infectious disease, or an autoimmune disease.

72. The method of any one of claims 69-71, wherein the subject is a human.

73. The method of any one of claims 69 to 72 wherein about 2 ug of saponin is delivered to the subject per dose.

74. The method of claim 73 wherein about 5 ug of GLA is delivered to the subject per dose.

75. The method of any one of claims 69 to 72 wherein about 4 ug of saponin is delivered to the subject per dose.

76. The method of claim 75 wherein about 10 ug of GLA is delivered to the subject per dose.

77. The formulation or compositions of any of the preceding claims wherein the formulations and compositions are not for use in the treatment of west nile virus.

78. The formulation or compositions of any of the preceding claims wherein the formulations and compositions are mixed with an antigen and the antigen is not associated with or derived from west nile virus.

79. The formulation or compositions of any of the preceding claims wherein the formulations and compositions are for use in the treatment of TB, HIV or malaria.

80. The formulation or compositions of any of the preceding claims wherein the formulations and compositions are mixed with an antigen and the antigen is associated with or derived from TB, HIV or malaria.

81. The formulation or composition of claim 80 wherein the antigen is ID93, ID91, or ID97.

82. A method of manufacturing saponin-containing liposomal formulations of any one of the preceding claims comprising mixing the saponin with pre-formed sterol-containing liposomes.

83. The method of claim 82 wherein the saponin is QS21 and the crude saponin mixture Quil A is purified to obtain the saponin.

84. The method of any one of claim 82 or 83 wherein the saponin is solubilized into buffer prior to mixing with liposomes.

85. The method of any one of claims 82 to 84 wherein the pre-formed sterol-containing liposomes are prepared by mixing the phospholipid and the sterol and reducing the particle size of the resultant liposomes via high pressure homogenization.
